(19) United States
(12) Patent Application Publication
(10) Pub. No.: US 2020/0330541 A1
(43) Pub. Date: Oct. 22, 2020

(54) COMPOSITIONS AND METHODS FOR MODULATING ENDOTHELIAL CELL MIGRATION AND ANGIOGENESIS

(71) Applicant: University of Maine System Board of Trustees, Orono, ME (US)

(72) Inventors: Dorothy Klimis-Zacas, Orono, ME (US); Panagiotis Tsakiroglou, Orono, ME (US)

(73) Assignee: University of Maine System Board of Trustees, Orono, ME (US)

(21) Appl. No.: 16/759,827

(22) PCT Filed: Nov. 15, 2018
(86) PCT No.: PCT/US2018/061197

§ 371 Date: Apr. 28, 2020

Related U.S. Application Data
(60) Provisional application No. 62/587,113, filed on Nov. 16, 2017.

(51) Int. Cl. A61K 36/45; A61P 17/02; A61P 9/00; A61K 31/216

(52) U.S. Cl. CPC A61K 36/45; A61K 31/216; A61P 9/00; A61P 17/02

(57) ABSTRACT

Materials and methods for modulating endothelial cell migration, angiogenesis, and gene expression of VEGF, AKT1, RAC1, RHOA, and eNOS are described.

### BACKGROUND

One fundamental process in cell morphogenesis, immune function, physiology, development, regeneration, and disease is cell migration. The wound healing process involves hemostasis, inflammation, cell differentiation, proliferation, and cell migration, which promotes angiogenesis and finally tissue remodeling. Since wound healing is a cellular response to injury, many cell types are involved in this complex biological phenomenon including endothelial cells, stimulated and coordinated to perform a balanced wound healing process. Several factors can affect wound healing and can be classified as local or systemic. Some of the systemic factors are: age, gender, stress, alcohol, smoking, malnutrition, obesity, and diseases. Chronic diseases such as diabetes lead to impaired wound healing which is a result of unbalanced angiogenesis. Reduced blood flow to the extremities, and a decrease in endothelial cell proliferation and angiogenesis, result in improper response of diabetic patients to injury.

It would be advantageous to develop new compositions and methods for modulating endothelial cell migration and angiogenesis.

### SUMMARY

Provided is a method of modulating endothelial cell migration, the method comprising contacting endothelial cells with an effective amount of an extract of berries from the Vaccinium family to modulate endothelial cell migration. In certain embodiments, the berries are blueberries.

In certain embodiments, the extract is a phenolic-rich fraction. In particular embodiments, the phenolic-rich fraction includes chlorogenic acid. In particular embodiments, the effect is increased cell migration. In particular embodiments, the effective amount of the extract is a concentration ranging from about 0.001 μg/mL to about 500 μg/mL. In particular embodiments, the effective amount of the extract is a concentration ranging from about 0.002 μg/mL to about 120 μg/mL. In particular embodiments, the effective amount of the extract is a concentration of about 60 μg/mL.

In certain embodiments, the extract is an anthocyanin-rich fraction. In particular embodiments, the effect is inhibited cell migration. In particular embodiments, the effective amount of the extract is a concentration ranging from about 10 μg/mL to about 200 μg/mL. In particular embodiments, the effective amount of the extract is a concentration of about 60 μg/mL.

In certain embodiments, the extract is a phenolic-rich fraction combined with an anthocyanin-rich fraction. In particular embodiments, the effect is increased cell migration. In particular embodiments, the effective amount of the extract is a concentration ranging from about 1 μg/mL:1 μg/mL of anthocyanin-rich fraction:phenolic-rich fraction to about 200 μg/mL:200 μg/mL of anthocyanin-rich fraction:phenolic-rich fraction. In particular embodiments, the effective amount of the extract is a concentration ranging from about 8 μg/mL:8 μg/mL of anthocyanin-rich fraction:phenolic-rich fraction to about 60 μg/mL:60 μg/mL of anthocyanin-rich fraction:phenolic-rich fraction.

In certain embodiments, the extract is an ethyl acetate soluble fraction. In certain embodiments, the extract is a methanol soluble fraction. In certain embodiments, the extract is from V. angustifolium. In certain embodiments, the endothelial cells are human endothelial cells. In certain embodiments, angiogenesis or wound healing is modulated.

Further provided is a method of modulating angiogenesis, the method comprising contacting endothelial cells with an effective amount of an extract of berries from the Vaccinium family and modulating angiogenesis.

Further provided is a method of modulating RAC1 or RHOA, the method comprising contacting cells with an effective amount of an extract of berries from the Vaccinium family and modulating RAC1 or RHOA gene expression in the cells. In certain embodiments, the cells are endothelial cells. In certain embodiments, the cells are human endothelial cells. In certain embodiments, the extract comprises a phenolic-rich fraction, and RAC1 gene expression is increased or RHOA gene expression is increased. In particular embodiments, the effective amount is a concentration ranging from about 0.001 μg/mL to about 200 μg/mL. In particular embodiments, the effective amount is about 0.002 μg/mL, about 60 μg/mL, or about 120 μg/mL. In certain embodiments, the extract comprises an anthocyanin-rich fraction, and RAC1 gene expression is increased or RHOA gene expression is increased. In particular embodiments, the effective amount is a concentration ranging from about 10 μg/mL to about 200 μg/mL. In particular embodiments, the effective amount is about 60 μg/mL. In certain embodiments, the extract comprises a combination of an anthocyanin-rich fraction and a phenolic-rich fraction, and RAC1 expression is increased or RHOA gene expression is increased. In particular embodiments, the effective amount is a concentration ranging from about 1 μg/mL or about 100 μg/mL (at a 1:1 ratio of anthocyanin-rich fraction to phenolic-rich fraction). In particular embodiments, the effective amount is about 8 μg/mL or about 60 μg/mL (at a 1:1 ratio of anthocyanin-rich fraction to phenolic-rich fraction).

Further provided is a method of modulating gene expression of eNOS in cells, the method comprising contacting cells with an effective amount of an extract from a berry of the Vaccinium family and modulating gene expression of eNOS in the cells. In certain embodiments, the cells are endothelial cells. In certain embodiments, the cells are human endothelial cells. In certain embodiments, the extract comprises an anthocyanin-rich fraction, and the gene expression of eNOS is decreased. In particular embodiments, the effective amount is a concentration ranging from about 10 μg/mL to about 200 μg/mL. In particular embodiments, the effective amount is about 60 μg/mL. In certain embodiments, the extract comprises a combination of an anthocyanin-rich fraction and a phenolic-rich fraction, and the gene expression of eNOS is decreased. In particular embodiments, the effective amount is a concentration ranging from about 10 μg/mL to about 200 μg/mL (at a 1:1 ratio of anthocyanin-rich fraction to phenolic-rich fraction). In particular embodiments, the effective amount is about 8 μg/mL (at a 1:1 ratio of anthocyanin-rich fraction to phenolic-rich fraction). In particular embodiments, the effective amount is about 60 μg/mL (at a 1:1 ratio of anthocyanin-rich fraction to phenolic-rich fraction).

Further provided is a method of modulating gene expression of AKT1 in cells, the method comprising contacting cells with an effective amount of an extract from a berry of the Vaccinium family and modulating gene expression of AKT1 in the cells. In certain embodiments, the cells are endothelial cells. In certain embodiments, the cells are human endothelial cells. In certain embodiments, the extract comprises a phenolic-rich fraction, and the gene expression of AKT1 is increased. In particular embodiments, the effective amount is a concentration ranging from about 0.001 μg/mL to about 100 μg/mL. In particular embodiments, the effective amount is about 0.002 μg/mL. In particular embodiments, protein expression of AKT1 is increased. In certain embodiments, the extract comprises an anthocyanin-rich fraction, and the gene expression of AKT1 is decreased. In particular embodiments, the effective amount is a concentration ranging from about 1 μg/mL to about 200 μg/mL. In particular embodiments, the effective amount is about 60 μg/mL. In certain embodiments, the extract comprises a combination of a phenolic-rich fraction and an anthocyanin-rich fraction, and the gene expression of AKT1 is decreased. In particular embodiments, the effective amount is a concentration ranging from about 1 μg/mL to about 200 μg/mL (at a 1:1 ratio of anthocyanin-rich fraction to phenolic-rich fraction). In particular embodiments, the effective amount is about 8 μg/mL (at a 1:1 ratio of anthocyanin-rich fraction to phenolic-rich fraction). In particular embodiments, the effective amount is about 60 μg/mL (at a 1:1 ratio of anthocyanin-rich fraction to phenolic-rich fraction), and protein expression of AKT1 is increased.

Further provided is a method of modulating gene expression of VEGF in cells, the method comprising contacting cells with an effective amount of an extract from a berry of the Vaccinium family and modulating gene expression of VEGF in the cells, wherein the extract comprises a phenolic-rich fraction. In certain embodiments, the cells are endothelial cells. In certain embodiments, the cells are human endothelial cells. In certain embodiments, VEGF gene expression is increased. In certain embodiments, the effective amount is a concentration ranging from about 0.001 μg/mL to about 100 μg/mL. In certain embodiments, the effective amount is about 0.002 μg/mL. In certain embodiments, the effective amount is about 60 μg/mL. In certain embodiments, the extract further comprises an anthocyanin-rich fraction. In particular embodiments, the effective amount is a concentration ranging from about 1 μg/mL to about 100 μg/mL. In particular embodiments, the effective amount is about 60 μg/mL.

### BRIEF DESCRIPTION OF THE DRAWINGS

FIG. 1: HPLC profile analysis of the individual ACN (panel A) and PA (panel B) fractions of wild blueberry (V. angustifolium) powder.

FIG. 2: Percent reduction of alamarBlue reagent after twenty-four hours of treatment with the ACN fraction. Treatment with ACNs at 1000 μg/mL inhibited endothelial cell proliferation rate compared to the control (*: p≤0.05).

FIG. 3: Percent reduction of alamarBlue reagent after twenty-four hours of treatment with the PA fraction.

FIG. 4: Endothelial cells treated with ACNs. Treatment with ACNs at 60 μg/mL inhibited endothelial cell migration rate compared to the control (*: p≤0.05).

FIG. 5: Endothelial cells treated with PAs. Treatment with PAs at 0.002 μg/mL, 60 μg/mL (****: p≤0.0001), and 120 μg/mL (**: p≤0.01) increased endothelial cell migration rate compared to control.

FIG. 6: Endothelial cells treated with combinations of both ACNs and PAs. Treatment with ACNs:PAs 8 μg/mL:8 μg/mL (*: p≤0.05) and ACNs:PAs 60 μg/mL:60 μg/mL (***: p≤0.001) increased endothelial migration rate compared to control.

FIG. 7: Summary of all treatments (ACNs 60 μg/mL, PAs 60 μg/mL, and combination of ACNs:PAs 60 μg/mL:60 μg/mL) that altered endothelial cell migration rate. Captured frames from time-lapse video from time-points at 0 h, 2 h, 4 h, 6 h, and 8 h.

FIG. 8A: Gene expression for RAC1 increased compared to the control for all tested concentrations two hours after treatment, ACNs 60 μg/mL (**: p≤0.01), PAs at 0.002 μg/mL (****: p≤0.0001), 60 μg/mL (***: p≤0.0001), and 120 μg/mL (**: p≤0.0001), and ACNs:PAs at 8 μg/mL and 60 μg/mL (**: p≤0.0001).

FIG. 8B: Gene expression for RHOA increased for all tested concentrations two hours after treatment, ACNs 60 μg/mL (***: p≤0.001), PAs at 0.002 μg/mL (***: p≤0.001), 60 μg/mL (***: p≤0.001), and 120 μg/mL (**: p≤0.01), and ACNs:PAs 8 μg/mL and 60 μg/mL (***: p≤0.001) compared to control.

FIG. 9A: Fold change western blot analysis of total RAC1 after acute treatment (two hours) of ACNs, PAs, and ACNs:PAs. Inhibition of RAC1 levels was documented with PAs at 0.002 μg/mL (*: p≤0.05) and increased levels of RAC1 were documented with ACNs:PAs at 8 μg/mL (**: p≤0.05) after 2 hours of treatment.

FIG. 9B: Fold change western blot analysis of RHOA with PAs at 0.002 μg/mL (**: p≤0.01), PAs at 60 μg/mL (*: p≤0.05), and ACNs:PAs at 8 μg/mL (*: p≤0.05) after 2 hours of exposure.

FIGS. 10A-10B: Images showing untreated HUVECs (FIG. 10A) and HUVECs treated with 60 μg/mL of ACNs (FIG. 10B). Parameters of analysis: number of meshes, total meshes area, number of nodes, number of master junctions, and total master segment length.

FIG. 11: Parameters of endothelial tube formation integrity after cells treated with ACNs at 60 μg/mL. Treatment with ACNs at 60 μg/mL documented decreased number of meshes (*: p≤0.05) and area of meshes (**:p≤0.01) compared to control.

FIGS. 12A-12C: Images of untreated HUVECs (FIG. 12A), HUVECs treated with PAs at 0.002 μg/mL (FIG. 12B), and HUVECs treated with PAs at 60 μg/mL (FIG. 12C).

FIG. 13: Parameters of endothelial tube formation integrity after cells treated with PAs at 0.002 μg/mL, 60 μg/mL, and 120 μg/mL. PAs at 0.002 μg/mL increased all the parameters measured for endothelial tube formation compared to the control. PAs at 60 μg/mL increased all the parameters but one (the number of the master junctions) compared to the control.

FIGS. 14A-14C: Images of untreated HUVECs (FIG. 14A), HUVECs treated with ACNs:PAs at 8 μg/mL (FIG. 14B), and HUVECs treated with ACNs:PAs at 60 μg/mL (FIG. 14C).

FIG. 15: Parameters of endothelial tube formation integrity after cells treated with a combination of both ACNs and PAs at 8 μg/mL and 60 μg/mL. ACNs:PAs at 60 μg/mL increased the total mesh area (****:p≤0.0001) and the total master segment length (**:p≤0.01) compared to the control.

FIGS. 16A-16D: Summary of three different treatments at the same concentration. FIG. 16A shows the control. FIG. 16B shows ACNs at 60 μg/mL. FIG. 16C shows PAs at 60 μg/mL. FIG. 16D shows ACNs:PAs at 60 μg/mL.

FIG. 17: Values reported as fold change corrected to GAPDH housekeeping gene. (*:p≤0.05) (**:p≤0.01) (***:p≤0.001) (****:p≤0.0001).

FIG. 18: Western blot analysis for AKT1. (**:p≤0.01) (***:p≤0.001).

FIG. 19: Western blot analysis for eNOS.

FIG. 20: Western blot analysis for VEGF.

### DETAILED DESCRIPTION

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are described herein. All references cited herein, including books, journal articles, published U.S. or foreign patent applications, issued U.S. or foreign patents, and any other references, are each incorporated by reference in their entireties, including all data, tables, figures, and text presented in the cited references.

All publications mentioned in this description, are hereby incorporated in their entirety as if fully set forth herein to form part of the description.

Numerical ranges, measurements and parameters used to characterize the invention—for example, angular degrees, quantities of ingredients, polymer molecular weights, reaction conditions (pH, temperatures, charge levels, etc.), physical dimensions and so forth—are necessarily approximations; and, while reported as precisely as possible, they inherently contain imprecision derived from their respective measurements. Consequently, all numbers expressing ranges of magnitudes as used in the specification and claims are to be understood as being modified in all instances by the term “about.” All numerical ranges are understood to include all possible incremental sub-ranges within the outer boundaries of the range. Thus, a range of 30 to 90 units discloses, for example, 35 to 50 units, 45 to 85 units, and 40 to 80 units, etc. Unless otherwise defined, percentages are wt/wt %.

The present disclosure relates generally compounds for modulating angiogenesis and, in particular, to specific extracts of the Vaccinium family that are capable of inhibiting or increasing endothelial cell migration, angiogenesis, and/or gene expression of eNOS, RAC1, RHOA, VEGF, or AKT1.

“Blueberry” is a general name for blue-colored berries of the Vaccinium family, but a number of different species may qualify as “blueberries” for purposes of this disclosure. Specifically, bilberries (European Blueberries or Vaccinium myrtillus) and the so-called “American” blueberries (various plants in the Vaccinium family) are all encompassed.

The “phenolic fraction” or “phenolic-rich fraction” or “phenolic acid fraction” (PA) described herein comprises mainly chlorogenic acid and is a fraction soluble in and extracted with ethyl acetate. The “anthocyanin-rich fraction” or “anthocyanin fraction” (ACN) contains a mixture of mainly anthocyanins that are soluble and extracted with methanol. The protocols followed for the extraction of the bioactive PA and ACN compounds are described in detail by in Del Bo′ C, Cao Y, Roursgaard M, Riso P, Porrini M, Loft S, et al., Anthocyanins and phenolic acids from a wild blueberry (Vaccinium angustifolium) powder counteract lipid accumulation in THP-1-derived macrophages, Eur J Nutr 2015, doi:10.1007/s00394-015-0835-z, which is incorporated herein by reference in its entirety. However, other methods of obtaining phenolic-rich fractions and anthocyanin-rich fractions are encompassed within the scope of the present disclosure. Determination of total phenolic concentration may be conducted, for instance, by the Folin-Ciocalteu method. For the determination of the anthocyanin fraction concentration, a pH differential method may be used. However, other methods are possible and encompassed within the scope of the present disclosure.

The lowbush blueberry (Vaccinium angustifolium) has been ranked as one of the richest food sources of bioactive compounds (polyphenols) such as anthocyanins (ACNs) and phenolic acids (PAs) generally found in fruits and vegetables. The antioxidant activity of wild blueberries is a result of anthocyanins, procyanidins, chlorogenic acid, and other phenolic compounds. ACNs from wild blueberries are primarily composed of delphinidin, malvidin, petunidin, cyanidin, and peonidin. Their phenolic content is often influenced by growing practices, location, and harvesting methods. Scientists have documented that a person on average can consume 180-215 mg/day of ACNs with concentrations in the plasma in the pmol/L-nmol/L range.

In vivo and in vitro studies have documented the beneficial effects of wild blueberry (Vaccinium angustifolium) consumption on inflammation and cardiovascular disease (CVD) as well as many other chronic diseases. However, little is known on the effect(s) of single ACNs and PAs and fractions from different berries on cell migration, angiogenesis, and wound healing.

Single ACNs such as nasunin ((Delph inidin-3-(p-coumaroylrutinoside)-5-glucoside)), a major component of the anthocyanin pigment in eggplant, has been found to inhibit HUVEC proliferation rate at 200 μM, 100 μM, and 50 μM, and Delphinidin (Dp) has been found to increase vascular endothelial growth factor (VEGF)-induced tube formation of HUVECS. Diet-delivered polyphenols (apigenin, delphinidin, ellagic acid, and epigallocatechin-3 gallate) inhibited endothelial cell migration, proliferation, and tubulogenesis through the JAK/STAT3 and MAPK signaling pathways. The inhibitory effect of ACNs (pelargonidin and its glucoside-conjugated form, pelargonidin-3-glucoside (P3G)) on cell proliferation and smooth muscle cell migration has been documented, with FAK being a significant molecular target of ACNs. ACNs extracted from black soybeen seeds coats have been found to stimulate wound healing in chronic wounds by reducing the inflammatory state of the wound.

Evidence documents the importance of small G proteins in cell motility. RHOA, RAC, and CDC42 are known members of the RHO family and they play a key role in the actin cytoskeleton. During cell migration, RAC is involved in the formation of lamellipodia at the leading edge of the tip/migrating cells, and RHOA is a regulator of actin stress fibers and is required during focal adhesions. In vitro (human microvascular endothelial cells and HUVECs) and in vivo (C57BL/6 mice) studies have documented the role of berry extracts and PAs (gallic acid from red raspberries) on cell migration and angiogenesis promoting or inhibiting expression of RHO GTPases, receptors (VEGFR2/NRP1), and other molecules (AKT, ERK 1/2, VEGFA, and p38) involved in molecular pathways controlling those functions.

Since there is paucity of research on the effects of anthocyanin and phenolic acids on endothelial cell migration, the effect of ACN and PA fractions and their combination (ACNs:PAs) from wild blueberry powder (Vaccinum angustifolium) was investigated to determine whether they operate to alter cell migration, specifically affecting proliferation rate of the endothelial cells and the speed of endothelial cell migration after acute exposure to the above compounds. Changes in gene expression and protein levels of RAC1 and RHOA proteins, critical for cell migration, were also evaluated, as were changes in gene expression of VEGF, eNOS, and AKT1. As described in the examples herein, it has been found that endothelial cell migration and angiogenesis are differentially modulated based on the bioactive fractions in a concentration-dependent manner. Thus, in accordance with the present disclosure, provided herein are compositions and methods involving extracts from berries of the Vaccinum family that are useful for modulating endothelial cell migration, angiogenesis, or gene expression of RAC1, RHOA, VEGF, eNOS, or AKT1.

Pharmaceutical compositions of the present disclosure comprise an effective amount of a blueberry extract (or extract from a berry of the Vaccinum family), and/or additional agents, dissolved or dispersed in a pharmaceutically acceptable carrier. The phrases “pharmaceutical” or “pharmacologically acceptable” refer to molecular entities and compositions that produce no adverse, allergic, or other untoward reaction when administered to an animal, such as, for example, a human. The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it need to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a composition herein and/or additional agent is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally (U.S. Pat. Nos. 6,753,514, 6,613,308, 5,466,468, 5,543,158; 5,641,515; and 5,399,363 are each specifically incorporated herein by reference in their entirety).

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In some cases, the form may be sterile and may be fluid to the extent that easy injectability exists. It may be stable under the conditions of manufacture and storage and may be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In some cases, it may be desirable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed are known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Sterile injectable solutions are prepared by incorporating the compositions in the required amount in the appropriate solvent with various other ingredients enumerated above, as required, followed by filtered sterilization. Generally, dispersions are prepared by incorporating the various sterilized compositions into a sterile vehicle which contains the basic dispersion medium and the required other ingredients from those enumerated above. In the case of sterile powders for the preparation of sterile injectable solutions, some methods of preparation are vacuum-drying and freeze-drying techniques which yield a powder of the active ingredient plus any additional desired ingredient from a previously sterile-filtered solution thereof. A powdered composition is combined with a liquid carrier such as, but not limited to, water or a saline solution, with or without a stabilizing agent.

In other embodiments, the compositions may be formulated for administration via various miscellaneous routes, for example, topical (i.e., transdermal) administration, mucosal administration (intranasal, vaginal, etc.), and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and luarocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroethylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation is composed of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age and weight, as well as the severity and response of the symptoms.

In particular embodiments, the compositions described herein are useful for modulating endothelial cell migration, angiogenesis, or gene expression of RAC1, RHOA, VEGF, eNOS, or AKT1. In some embodiments, the compositions are administered to endothelial cells in a human or animal subject.

It is further envisioned that the compositions and methods described herein can be embodied in the form of a kit or kits. A non-limiting example of such a kit is a kit for modulating endothelial cell migration, angiogenesis, or gene expression of RAC1, RHOA, VEGF, eNOS, or AKT1, the kit comprising a phenolic-rich fraction of an extract from a berry of the Vaccinum family and an anthocyanin-rich fraction of an extract from a berry of the Vaccinum family, where the containers may or may not be present in a combined configuration. Many other kits are possible. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive or CD-ROM. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate

### Examples

### Example I—Phenolic and Anthocyanin Fractions from Wild Blueberries (V. angustifolium) Differentially Modulate Endothelial Cell Migration

These examples demonstrate that anthocyanin (ACN) and phenolic acid (PA) fractions and their combination (ACNs:PAs) from wild blueberry powder (Vaccinum angustifolium) affects the proliferation rate and speed of endothelial cell migration in human umbilical vein endothelial cells. Concentrations (0.001 μg/mL-1000 μg/mL for ACNs and 0.001 μg/mL-500 μg/mL for PAs) were tested for cell proliferation and cytotoxicity using the alamarBlue assay (Life Technologies, DAL1025). Cell migration was monitored using the IBIDI Culture-Insert (Ibidi, Munich, Germany). The treated and untreated (control) cells were observed under an inverted phase-contrast optical microscope (Nikon TS100) for up to 24 h. The speed of closure was calculated as the cell migration rate (v_{migration} in μm/hr) and analyzed with TScratch software. Treatment with ACNs at 60 μg/mL inhibited endothelial cell migration rate (p≤0.05) while PAs at 0.002 μg/mL (p≤0.0001), 60 μg/mL (p≤0.0001), and 120 μg/mL (p≤0.01) increased endothelial cell migration rate compared to control. Moreover, treatment with ACNs:PAs at 8 μg/mL:8 μg/mL (p≤0.05) and ACNs:PAs at 60 μg/mL:60 μg/mL (p≤0.001) increased endothelial cell migration. The results indicate that endothelial cell migration is differentially modulated based on the type of extract and is concentration-dependent.

Anthocyanin (ACN) and phenolic acid (PA) fractions and their combination (ACNs:PAs) from wild blueberry powder (Vaccinum angustifolium) affects the proliferation rate and speed of endothelial cell migration in human umbilical vein endothelial cells.

Materials and Methods

Cell Culture

Human umbilical vein endothelial cells (HUV-EC-C [HUVEC] (ATCC® CRL-1730™)) were purchased from the American Type Culture Collection (ATCC®) Manassas, Va., USA. Human umbilical vein endothelial cells were maintained in F-12K medium (Kaighn's modification of Ham's F-12 medium) (ATCC® 30-2004™) with 10% fetal bovine serum (FBS) (ATCC® 30-2020™) and 1% penicillin-streptomycin solution (ATCC® 30-2300™), heparin 0.1 mg/mL (Sigma, H3149), and endothelial cell growth supplement (ECGS) 0.03 mg/mL (Sigma E2759). The culture vessel for the growth of the cells were the Corning® T-75 flask (catalog #3276). The culture conditions for the cell line was air 95%, carbon dioxide (CO₂) 5%, 90% of relative humidity (RH) and temperature of 37° C.

Extraction and Analysis of ACNs and PA Fractions from Wild Blueberry Powder

Wild blueberries (WB) were provided as a composite by Wyman's (Cherryfield, Me., USA) and processed following standard procedures to obtain a freeze-dried powder (FutureCeuticals, Momence, Ill., USA). Vacuum-packed plastic bags with the wild blueberry powder were stored at −20° C. until use. The wild blueberry powder had a total content of 1.5% w/w of anthocyanins, with malvidin-3-galactoside and peonidin-3-glucoside being the most abundant forms. From the freeze-dried wild blueberry powder three fractions were isolated: 1. Phenolic-rich fraction (ethyl acetate soluble, containing mainly chlorogenic acid); 2. Anthocyanin-rich fraction (methanol soluble fraction, containing mainly anthocyanins); and 3. Water soluble fraction.

The extraction of ACNs and PAs from the WB powder was performed according to a previously described method. The protocol followed for the extraction of the bioactive compounds is described in detail in Del Bo′ C, Cao Y, Roursgaard M, Riso P, Porrini M, Loft S, et al., Anthocyanins and phenolic acids from a wild blueberry (Vaccinium angustifolium) powder counteract lipid accumulation in THP-1-derived macrophages, Eur J Nutr 2015, doi:10.1007/s00394-015-0835-z, which is incorporated herein by reference. Determination of total phenolic concentration was conducted by the Folin-Ciocalteu method. For the determination of the anthocyanin fraction concentration, a pH differential method was used.

The concentration of ACN and PA in the wild blueberry fraction was determined by HPLC. The system was composed of an Alliance Mod. 2695 (Water, Milford, Mass.) equipped with a mod. 2998 photodiode array detector (Waters).

Growth Curve

Calculating a growth curve for the HUVECs provides useful information for the growth characteristics of this cell line. For this experiment, the multiwell protocol was used. Six 6-well plates (Corning, Product #3335) were used. From each six-well plate, two wells were plated with 1×10⁴ cells/mL, two with 3×10⁴ cells/mL, and two with 1×10⁵ cells/mL. The six-well plates were placed in a humid incubator (humidity level 90%) with 5% CO₂ level and 37° C. temperature. After 24 h, one well from each cell density (1×10⁴ cells/mL, 3×10⁴ cells/mL, and 1×10⁵ cells/mL) was aspirated with 500 μl of TrypLE™ Express Enzyme (1×), containing no phenol red (LifeTechnologies 12604-013). A hemocytometer was used to count the live and dead cells from each well and Erythrosine B stain solution (ATCC® 30-2404™) was used to stain dead cells. The remaining wells from the six-well plate were returned to the incubator. Every 24 hs the same procedure was executed until the twelfth day. After plotting the cell concentration (y axis) versus days after subculture (x axis) the lag phase, log phase and plateau phase were calculated.

Cell Proliferation and Cytotoxicity Assay

The cell growth curve experiment provided information on the doubling time of the HUVECs. The anthocyanin and phenolic acid cytotoxicity assay was conducted so that the optimum concentration and exposure time of the active compounds was used for the rest of the experiments. Different concentrations (0.001 μg/mL-1000 μg/mL for ACNs and 0.001 μg/mL-500 μg/mL for the PAs) and different exposure times were tested (30 min, 1 h, 3 h, 6 h, 12 h, 24 h, 48 h, and 72 h) were tested. Cell proliferation and cytotoxicity assays were conducted by using the alamarBlue assay (Life Technologies, DAL1025). Before conducting the cell proliferation and cytotoxicity experiments, a standard curve of alamarBlue was generated. AlamarBlue is a very sensitive assay that can detect as few as 50 cells/96-well plate. Cells for both cell proliferation and cytotoxicity assays were measured by using Synergy 2 multiwell plate ready (Bio-Tek Instruments Inc., Winooski, Vt.) with excitation/emission (530 nm-560 nm/590 nm).

Cell Migration

The effect of ACN, PAs, and combinations of both fractions on cell migration was evaluated by the IBIDI Culture-Insert (Ibidi, Munich, Germany). The culture inserts were seeded with 5×10⁵ cells/mL and the final volume in each insert chamber was 70 μl. The IBIDI Culture-Insert was placed in the incubator for 24 h. After cells reached ≥90% confluence they were treated with the PA and ACN fractions and their combinations at concentrations were determined by the results of the cytotoxicity experiment (0.002 μg/mL, 8 μg/mL, 15 μg/mL, 60 μg/mL, and 120 μg/mL). Cells in the treated (ACN and PA fractions and combinations) and untreated (control, n=10 replicates) wells were observed under an inverted phase-contrast optical microscope (Nikon TS100) until endothelial cells fully migrated into the free area. At the end of each experiment the speed of closure was calculated as the cell migration rate (v_{migration} in μm/hr). Analysis was conducted with the TScratch software.

Gene Expression, Real-Time RT-PCT Analysis

Endothelial cells were cultured and maintained as previously described. HUVECs were treated with ACN and PA fractions and their combinations for 2 h and 6 h. mRNA was isolated using the RNeasy Kit (Qiagen) and DNase Diegstion (Qiagen) was used for RNA purification. QuantiTect Reverse Transcription Kit (Qiagen) was used for cDNA synthesis and removal of genomic DNA. A two-step RT-PCT was followed using a CFX96 (BioRad) PCT system. A 20 μl PCR reaction volume was performed using TaqMan gene expression master mix (Invitrogen) and TaqMan probes (Invitrogen): RHOA (Hs00357608_m1), RAC1 (Hs01902432_s1), and GAPDH (Hs99999905_m1).

Active GTPase Pull-Down Assay

For immunoprecipitation, cells were lysed using the Cell Signaling active RHO detection kit (8820) and active RAC1 detection kit (8815) following manufacturer's instructions, and treated with GTPyS (positive control) and GDP (negative control). Moreover, in the 1× Lysis/Binding/Wash Buffer, phenylmethylsulfonyl fluoride (PMSF) (Cell Signaling, 8553) was used at a concentration of 1 mM. Concentration of total protein was measured using BCA assay (Thermo Fisher, 23225). Immunoprecipitated materials and total protein samples were resolved by 4-20% mini-protean TGX stain-free protein gel (BioRab, 4568094). After transfer using Trans-Blot Turbo System (BioRad, 1704150), LF-PVDF membranes (BioRad, 1704274) were blotted with anti-RAC1 (1:1000, Cell Signaling, 8631) and anti-RHOA (1:667, Cell Signaling, 8789). Proteins were detected with antibodies specific for either mouse or rabbit IRDye® 800CW Goat anti-Mouse IgG, (1:15000, Li-COR, 925-32310) and IRDye® 800CW Goat anti-Rabbit IgG, (1:15000, Li-COR, 925-32211) using the Li-COR Odyssey imaging system (Li-COR Biosciences). All assays were repeated at least four times in independent experiments.

Statistical Analysis

Statistical analysis was performed using GraphPad Prism 6.0 software (GraphPad Software, San Diego, Calif.). For the cell migration experiments, the data were analyzed by one-way ANOVA. For post-hoc comparisons, Fisher's least significant difference (LSD) test was used. For the gene expression and western blot experiments, a two-tail Mann-Whitney U-test was performed to compare the control to ACNs (60 μg/mL), while one-way ANOVA was used for PA (0.002 μg/mL, 60 μg/mL, and 120 μg/mL) and combination (ACNs:PA) (8 μg/mL:8 μg/mL and 60 μg/mL:60 μg/mL) groups. For post-hoc comparisons, Fisher's least significant difference (LSD) test was used. All data in the graphs are expressed as mean+/−SEM. A p-value of <0.05 was considered significant. For the cell migration experiments, the control included 10 replicates (n+10) and each treatment group (ACNs, PAs, and ACNs:PAs) included 7 replicates (n=7), while for gene expression experiments the control included 10 replicates (n=10) and each treatment group (ACNs, PAs, and ACNs:PAs) included 10 replicates (n=10). Lastly, for IP experiments, the control included 4 replicates (n=4) and each treatment group (ACNs, PAs, and ACNs:PAs) included 4 replicates each (n=4).

Results

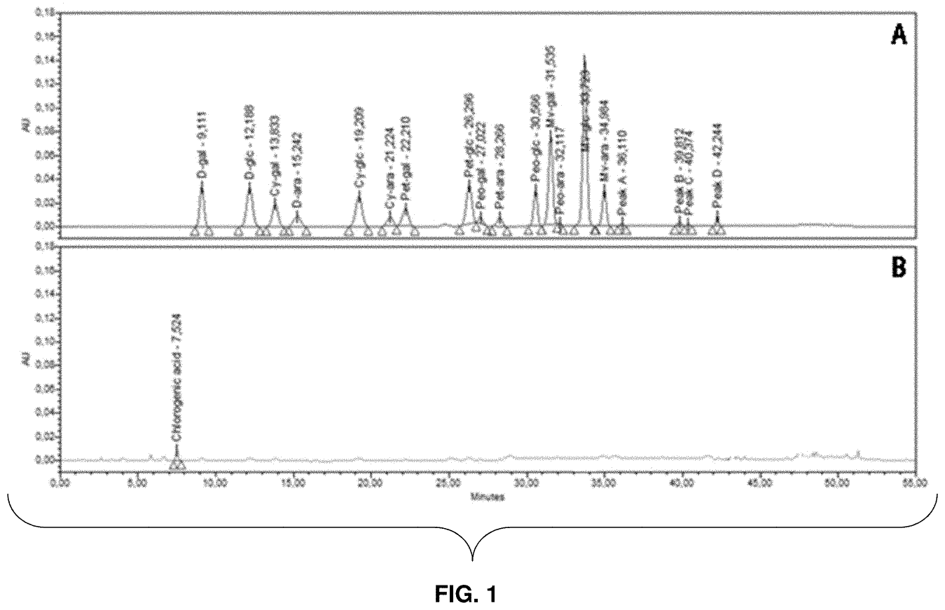

Analysis of the wild blueberry powder anthocyanin extract is reported in Table 1 and FIG. 1. FIG. 1 shows the HPLC profile analysis of the ACN fraction. The total ACN concentration was 45.11±0.35 mg/mL with 15 different ACNs detected. Malvidin glucosides were higher in concentration (26.5%) followed by malvidin galactoside (14.8%) while delfinidin glucoside (8.9%), petunidin glucoside (8.2%), and cyanidin glucoside (7.4%) followed. The PA fraction contained mainly chlorogenic acid (10.23±1.8 mg/mL) with traces of ferulic and caffeic acids (FIG. 1).

TABLE 1
Characterization of the ACN fraction extracted from
the wild blueberry powder (V. angustifolium)

| peak | name | % |
|---|---|---|
| 1 | D-gal | 6.4 |
| 2 | D-glc | 8.9 |
| 3 | Cy-gal | 4.8 |
| 4 | D-ara | 2.6 |
| 5 | Cy-glc | 7.4 |
| 6 | Pet-gal | 1.8 |
| 7 | Cy-ara | 4.1 |
| 8 | Pet-glc | 8.2 |
| 9 | Peo-gal | 1 |
| 10 | Pat-ara | 1.6 |
| 11 | Peo-glc | 5.3 |
| 12 | Mv-Gal | 14.8 |
| 13 | Peo-ara | 0.1 |
| 14 | Mv-glc | 26.5 |
| 15 | Mv-ara | 5.3 |
| A | Peak A | 0.2 |
| B | Peak B | 0.3 |
| C | Peak C | 0 |
| D | Peak D | 0.7 |
| | Total | 100 |

Cell Proliferation Assay

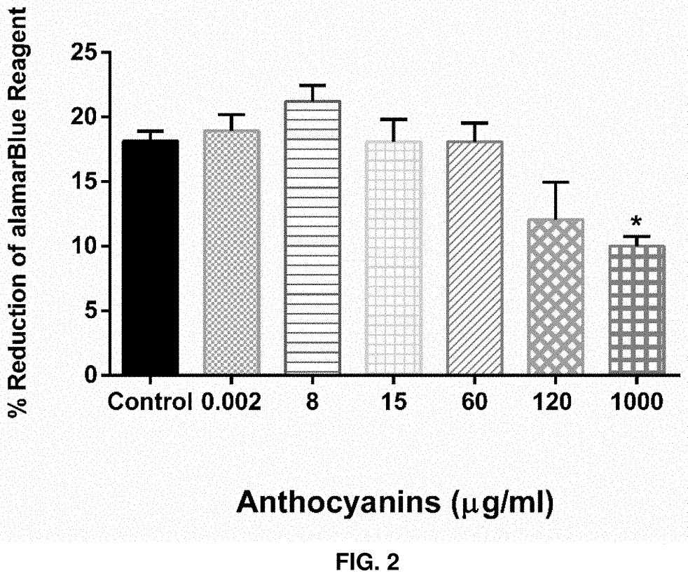

The HUVEC proliferation rate was calculated to seed the appropriate number of cells at the 96-well plate for the cell cytotoxicity assay. Quadruplicate samples were used for each different concentration of ACNs and PAs. Results after twenty-four hours of treatment with ACNs and PAs are presented in FIG. 2.

No significant differences were observed in HVUEC proliferation rates during the different exposure times tested (30 min, 1 h, 3 h, 6 h, 12 h, 24 h, 48 h, and 72 h) at the concentrations tested (0.001 μg/mL-1000 μg/mL for ACNs and 0.001 μg/mL-500 μg/mL for the PAs compared to the control. (FIG. 2.) The highest concentration of ACNs (1000 μg/mL) only documented inhibition of the proliferation rate of endothelial cells.

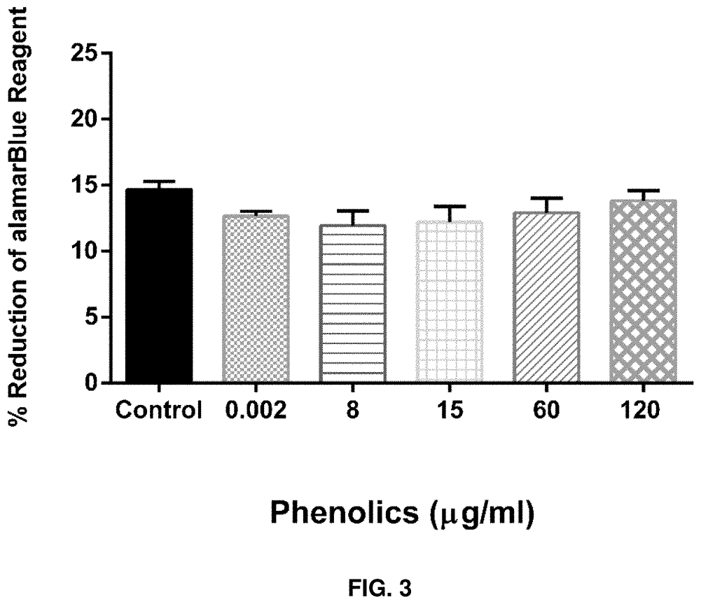

Similarly, exposure of HUVECs for twenty-four hours to the PA extract did not show any significant differences in HUVEC proliferation rate (FIG. 3).

Exposure of HUVECs to the above ACN and PA fractions concentrations for seventy-two hours documented no statistically significant difference on cell proliferation.

Inhibition of the proliferation rate of endothelial cells treated with high concentrations of ACNs (1000 μg/mL) compared to the control was documented twelve hours after exposure.

Cell Migration Assay

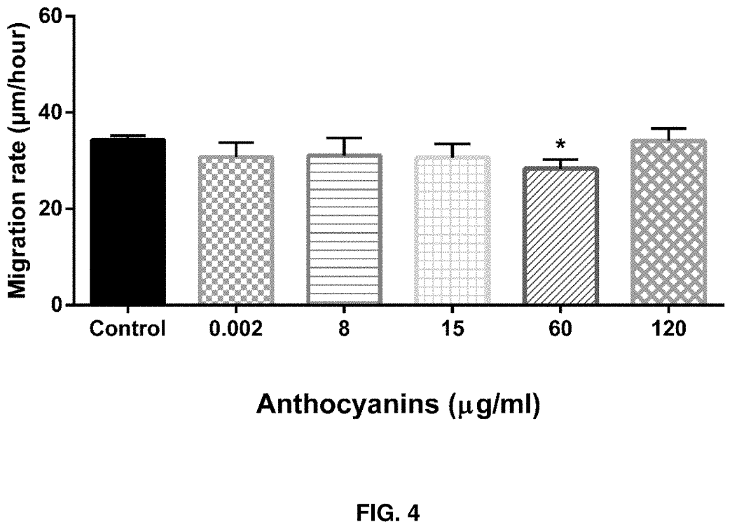

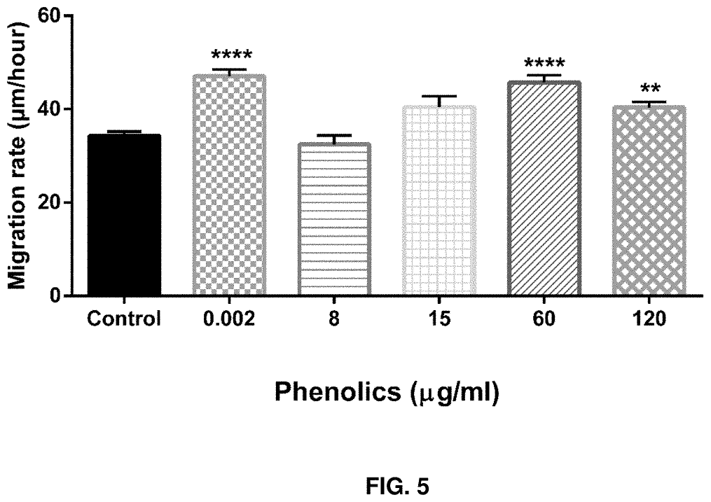

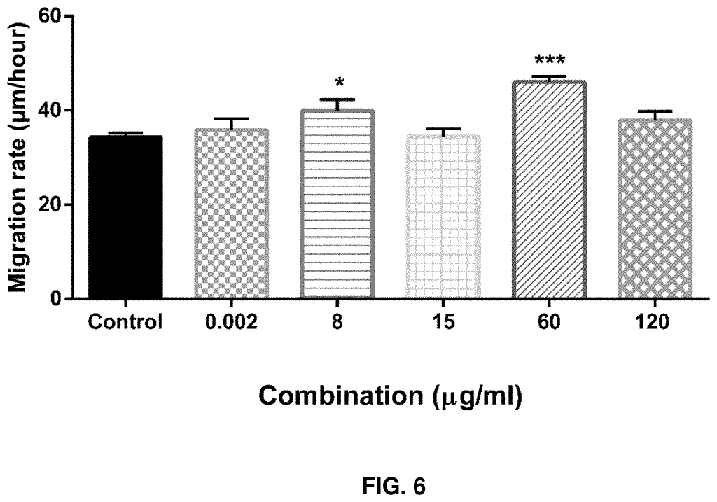

After determining the appropriate concentrations of ACN and PA fractions that were not cytotoxic to the HUVECs, the cell migration assay was performed to evaluate the effect of five concentrations (0.002 μg/mL, 8 μg/mL, 15 μg/mL, 60 μg/mL, and 120 μg/mL) of ACNs, PAs, and their combination (ACNs:PAs). During treatment with ACNs (FIG. 4), endothelial cell migration speed was reduced at the 60 μg/mL compared to control (p≤0.05). No statistically significant differences in cell migration were detected at ACN concentrations of 0.002 μg/mL, 8 μg/mL, 15 μg/mL, and 60 μg/mL. Endothelial cell migration was affected in the opposite way after treatment with PAs (FIG. 5). Treatment with PAs (FIG. 5) promoted endothelial cell migration. Exposure of the endothelial cells to 0.002 μg/mL, 60 μg/mL (p≤0.0001), and 120 μg/mL (p≤0.01) PAs significantly increased the speed of endothelial cell migration compared to control. Additionally, combination of both ACNs and PAs (ACNs:PAs) at 8 μg/mL:8 μg/mL and 60 μg/mL:60 μg/mL respectively, significantly increased endothelial cell migration rate compared to control (FIG. 6). Statistical differences among treatment groups were not detected at any of the other tested ACN:PA combinations.

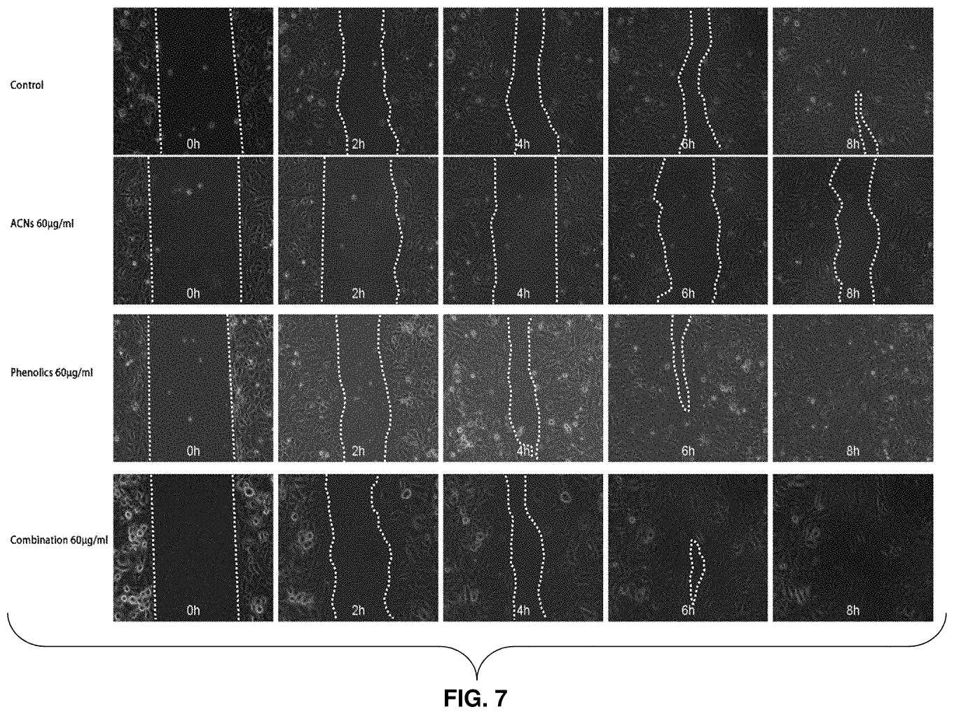

Visual depictions of cell migration from all treatments of ACNs, (60 μg/mL), PAs (60 μg/mL), and combination of both fractions (60 μg/mL:60 μg/mL) (FIG. 7) can be observed by frames from exposure of HUVECs at different time points (0 h, 2 h, 4 h, 6 h, and 8 h). In this montage in FIG. 7, pictures have been taken from the time-lapse video at the above-mentioned time-points and the cell migration border is marked with white dotted lines so that the progression of endothelial cell migration towards the empty area can be clearly observed.

Gene Expression and Immunoprecipitation (IP) Assays

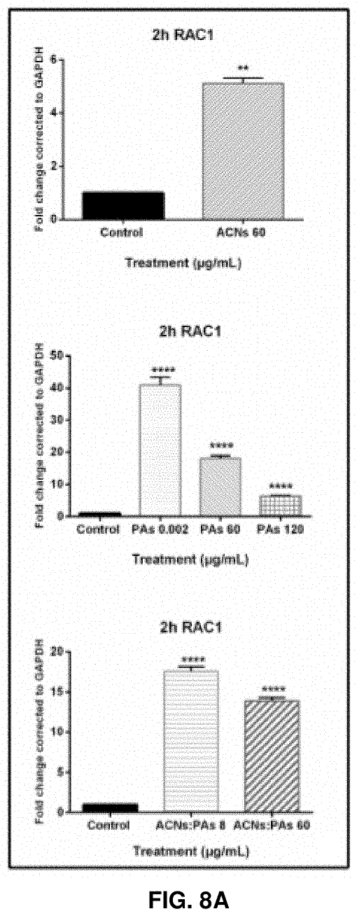

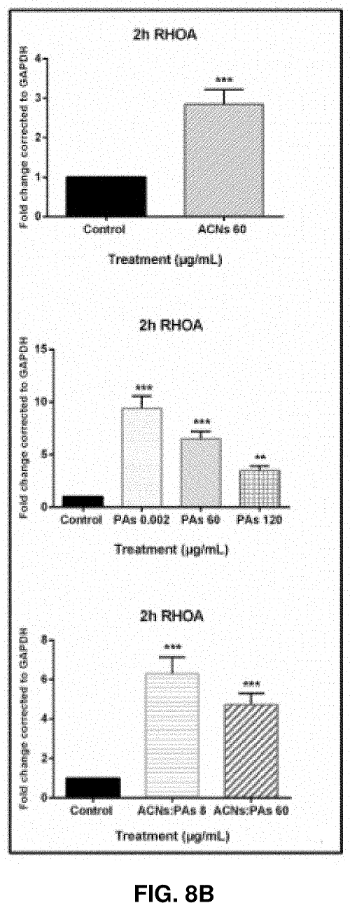

After determining the concentrations that documented a significant effect of endothelial cell migration, gene expression and IP experiments were important to reveal whether there was a change in RAC1 and RHOA genes and protein levels. Concentrations tested were ACNs at 60 μg/mL, PAs at 0.002 μg/mL, 60 μg/mL, and 120 μg/mL, and ACNs:PAs at 8 μg/mL and 60 μg/mL. Gene expression for RAC1 (FIG. 8A) and RHOA (FIG. 8B) was tested at two hours post treatment. After 2 hours of HVUEC exposure to ACNs, gene expression of RAC1 (FIG. 8A) increased almost fourfold compared to control. Similarly, exposure to PAs for 2 hours significantly upregulated RAC1 expression compared to control, at all concentrations tested. Gene expression for RHOA (FIG. 8B) also showed a similar pattern as RAC1.

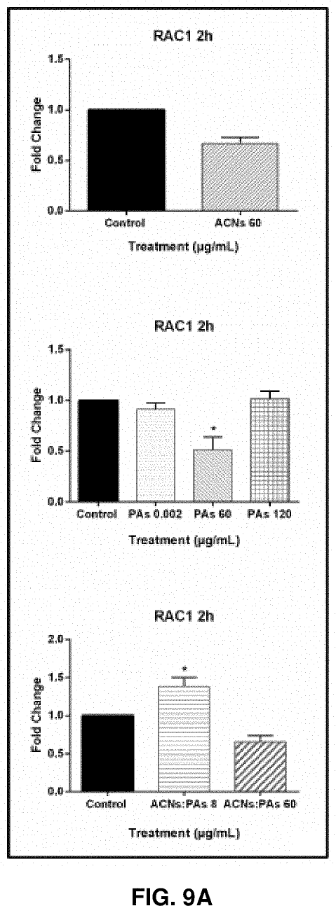

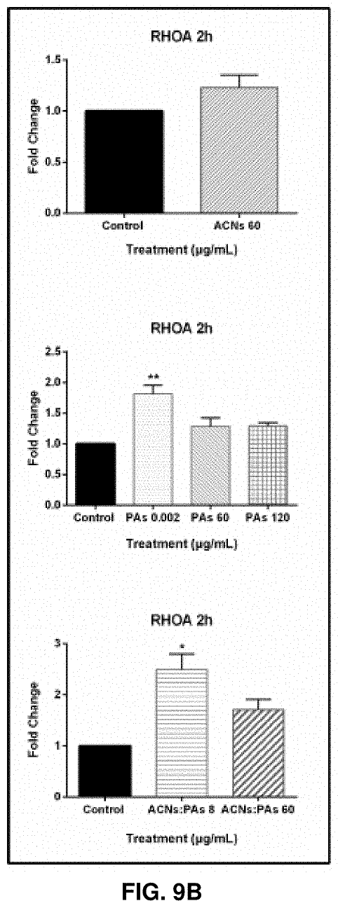

Quantitative western blot analysis of total RAC1 (FIG. 9A) documented inhibition of total RAC1 when cells were treated with PAs at 0.002 μg/mL, while total RAC1 increased at ACNs:PAs at 8 μg/mL after 2 hours of exposure only.

Quantitative western blot analysis of RHOA (FIG. 9B) documented increased levels of RHOA when cells were treated with PAs at 0.002 μg/mL (**: p≤0.01) and ACNs:PAs at 8 μg/mL (*: p≤0.05) 2 hours post treatment.

Discussion

The present example evaluated the effects of different concentrations of compounds, and their combinations, from wild blueberries on endothelial cell migration speed, gene expression, and protein synthesis of RAC1 and RHOA, as well as their effect on the proliferation rate of endothelial cells. This has demonstrated that different compounds (ACNs and PAs) extracted from wild blueberries can differentially modulate endothelial cell migration.

Endothelial cell migration is important for normal wound healing processes, tissue formation, and angiogenesis. Collective cell migration is a type of migration that plays a key role in wound healing. In vitro experiments with HUVECs have unraveled possible mechanisms of collective cell migration and the role of cadherin as well as the role of RAC1, RHOA, CDC43, and F-actin in the above process.

A reduction of cell proliferation was observed at 1000 μg/mL only, compared to control, at 12 hours after treatment. Under the experimental conditions, inhibition of cell migration was documented when HUVECs were exposed to the ACN wild blueberry extract at 60 μg/mL, and increased expression of RAC1 and RHOA was observed at 2 hours after exposure. In contrast, exposure of HUVECs to 0.002 μg/mL, 60 μg/mL, and at 120 μg/mL of the PA fraction, significantly increased speed of migration and gene expression of RAC1 and RHOA at 2 hours. The combination of both fractions simultaneously was also tested. The combination of fractions of ACNs:PAs at 8 μg/mL:8 μg/mL and 60 μg/mL:60 μg/mL significantly promoted endothelial cell migration, similar to the PA fraction. The major component of the PA fraction was chlorogenic acid, which is the most abundant polyphenol in the human diet.

Some studies have shown gallic, protocatechuic, syringic, and chlorogenic acids inhibit migration and angiogenesis processes by acting as cancer chemotherapeutic agents, and that using gallic acid inhibits cell motility, inhibits of NF-kB activity, and down regulates the PI3K/AKT pathway. In contrast, the PA fraction in this example containing primarily chlorogenic acid promoted endothelial cell migration and gene expression of RHOA and RAC1, molecules believed to be critical in the above process.

Without wishing to be bound by theory, it is believed that the differences in endothelial cell migration speed observed in this example when cells were treated with the same concentration, but different fractions, may be explained by factors such as chemical and structural differences among them, stability of the compounds, availability to HUVEC cell receptors, the level of oxidation, glycosylation, the ability to form polymeric molecules, and the existence of stereoisomers.

RHOA and RAC are important players during cell migration, with RHOA acting in the back of the cell while RAC1 acts in the front with CDC42. RHOA plays an important role in actin cytoskeleton formation and is involved in mechano-transduction through RHOA/RHO-kinase signaling. RAC1 also plays a significant role in endothelial function by controlling eNOS and the phosphorylation of proteins critical for cell to cell junction such as occludin, VE-cadherin, and b-catenin. In this example, gene expression of RAC1 and RHOA increased compared to the control after 2 hours of exposure with all treatments while western blot analysis of RAC1 was significantly reduced after exposure to 60 μg/mL of PAs, and was significantly induced after exposure to the 8 μg/mL combination. Finally, western blot analysis of total RHOA showed RHOA was significantly increased after 2 hours of exposure to 0.002 μg/mL PAs and 8 μg/mL combination. Even though the ACNs fraction significantly inhibited HUVEC cell migration speed, gene expression of RHOA and RAC1 was significantly increased. RHO GTPases are believed to be responsible for cytoskeleton changes. However, since the RHO family is composed of many members, there is a possibility that other members such as CDC42 may be responsible for the observations made. Without wishing to be bound by theory, it is believed that cadherin finger formation may also be important for cell-to-cell interaction between the leading and following cell, and RHOA is more important for the formation of cadherin fingers than RAC1, which is self-governing. It is also known that a critical activator of RAC1 is RHOG. In addition, shear stress can also act as a RAC1 activator.

Whereas previous studies have shown that caffeic acid at 0-100 μg/mL on smooth smuscle cells significantly decreased RAC1 protein synthesis after 24 hour exposure at 1 μmol/L and 100 μmol/L compared to untreated cells, and protocatechuic acid downregulated the Ras/Akt/NF-kB pathway by targeting RHOB, the PA fraction described in this example contained chlorogenic acid.

The present example examined the effects of a range of low and high concentrations of ACNs and PAs, obtained from wild blueberry fractions, on cell proliferation, endothelial cell migration, and gene expression of RAC1 and RHOA. Some of the concentrations used (0.002 μg/mL and 8 μg/mL) were close to the physiologically reported single compound concentrations that have been observed in the blood stream. For example, ACNs can be found in the blood stream at the concentration of 274 nM after consumption of foods rich in these bioactives. Research evaluating the fate of chlorogenic acids after coffee ingestion in healthy humans has documented a concentration of 385 μmol isolated from ileal fluid. The presence of four ACNs, delphinidin, 3-O-β-rutinoside (D3R), cyanidin 3-O-β-glucoside (C3G), in the plasma of healthy humans after consumption of black currant has been detected at concentrations of 73.7+/−35.0 nmol/L, 46.3+/−22.5 nmol/L, 22.7+/−12.4 nmol/L, and 5.0+/−3.7 nmol/L, respectively. A range of ACNs and PAs from 0.05 to 10 μg/mL has been used for lipid accumulation in macrophages. In this example, the concentrations of the extracts ranged from 9 nmol/L to 579 nmol/L for ACNs and 5.6 nmol/L to 338.6 nmol for PAs.

In sum, this example demonstrates that different fractions extracted from wild blueberries have a significant and differential effect on endothelial cell migration, which plays a key role in many physiological phenomena such as angiogenesis and wound healing. These differential effects are dose and compound dependent, and appear to be orchestrated by the modulation of RAC1 and RHOA, two proteins involved in cell motility.

The cytotoxic effect that different ACN concentrations have delivered either from dietary sources or commercially available individual compounds have been investigated previously. In the present example, the concentrations used to evaluate the cytotoxic effect of ACNs and PAs documented no significance compared to control.

A notable finding was that PAs at 0.002 μg/mL had a strong effect in increasing speed of cell migration. Additionally, PAs at 60 μg/mL and at 120 μg/mL had a similar effect on endothelial cell migration as the ones at 0.002 μg/mL. The combination of both fractions simultaneously was tested since. The combination of both anthocyanin and phenolic acid fractions at 8 μg/mL and 60 μg/mL induced endothelial cell migration speed. The combination of fractions at ACNs:PAs 8 μg/mL:8 μg/mL and ACNs:PAs 60 μg/mL:60 μg/mL has an effect similar to phenolic acids alone but not anthocyanins.

In this example, the major PA in the wild blueberry powder was chlorogenic acid. This example demonstrates that 0.002 μg/mL, 60 μg/mL, and 120 μg/mL PA can significantly increase speed of endothelial cell migration. Phenolic acids from wild blueberries were extracted and used in the experiments. Moreover, a broad range of concentrations were used to be able to cover a wider spectrum of the possible effect of PAs on endothelial cell migration.

### Example II—Wild Blueberry (V. angustifolium) Fractions (Anthocyanins and Phenolic Acids) Modulate In Vitro Angiogenesis in HUVECs

This example describes the effect of anthocyanin (ACN) and phenolic acid (PA) fractions, and their combinations (ACNs:PAs), from wild blueberry powder (Vaccinum angustifolium) on angiogenesis, gene expression, and protein level of AKT, eNOS, and VEGF associated with acute exposure to different concentrations of ACNs, PAs, and ACNs:PAs. Human umbilical vein endothelial cells (HUVECs) were used and ACN, PA, and ACNs:PAs at concentrations of 0.002 μg/mL, 8 μg/mL, 15 μg/mL, and 60 μg/mL were tested for endothelial tube formation (in vitro angiogenesis). Five parameters of angiogenesis network were analyzed: a. number of meshes; b. total meshes area; c. number of nodes; d. number of master junctions; and e. total master segment length.

Treatment with ACNs at 60 μg/mL resulted in decreased number of meshes (*: p≤0.05) and area of meshes (**: p≤0.01). PAs at 0.002 μg/mL increased all the parameters measured for endothelial tube formation. PAs at 60 μg/mL increased all the parameters but one, namely, the number of the master junctions. ACNs at 60 μg/mL decreased (compared to the control) the gene expression of AKT1 (**: p≤0.01) and eNOS (***: p≤0.001), but no effect was documented on VEGF. PAs at 0.002 μg/mL and 60 μg/mL increased AKT1 and PAs at 0.002 μg/mL and 60 μg/mL increased VEGF gene expression. ACNs:PAs at 8 μg/mL decreased gene expression of AKT1 and eNOS, but increased levels of gene expression for VEGF were documented. ACNs:PAs at 60 μg/mL decreased gene expression of eNOS; however, increased levels of gene expression for VEGF were documented. Protein levels of AKT1 were increased with PAs at 0.002 μg/mL (*: p≤0.05) and ACNs:PAs at 60 μg/mL (**: p≤0.01). These results indicate that tube network is differentially modulated based on the type of blueberry extract and is concentration-dependent.

Introduction

Blood vessels are responsible to carry oxygen to all organs of the human body. The growth of blood vessels from pre-existing ones is known as the process of angiogenesis. In cardiovascular biology there are three different types of blood vessel formation: angiogenesis, arteriogenesis, and vasculogenesis. In adults, angiogenesis initiated mainly from hypoxia-inducible factor (HIF)-1α expression. Arteriogenesis is the de novo formation of blood vessels. Finally, vasculogenesis is the in situ formation of blood vessels from vascular progenitor cells and circulating endothelial progenitor cells (EPCs). All three processes are subcategories of neovascularization that can occur in adults. Control of angiogenesis can act as a therapeutic tool. Nearly four decades ago it was hypothesized that inhibition of angiogenesis would be a way to treat human cancer effectively. Clinical trials have documented promising results that angiogenesis can be an important target for cancer and other diseases. Therapeutic angiogenesis can play a significant role in diseases such as ischemic disorders, but a better understanding of molecular pathways involve in angiogenesis is critical.

The lowbush blueberry (Vaccinium angustifolium) is one of the richest in flavonoids among fruits and berries. Even though wild blueberries are low in antioxidant vitamins and minerals, they are a rich source of bioactive compounds (polyphenols) such as anthocyanins and phenolic acids, as well as flavonoids, that are generally found in fruits and vegetables. The antioxidant activity of wild blueberries is a result of anthocyanins, procyanidins, chlorogenic acid, and other flavonoid compounds. Anthocyanins from wild blueberries are primarily composed of delphinidin, malvidin, petunidin, cyanidin, and peonidin. It is also known that phenolic content in these berries is often influenced by growing practices, location, and harvesting methods. Numerous studies have documented the beneficial effects of wild blueberry (Vaccinium angustifolium) consumption on inflammation and cardiovascular disease (CVD), as well as many other chronic diseases.

During cell proliferation and other important cellular functions such as cell migration, cell growth, and metabolism, AKT kinase plays a key role. It a member of AGC kinases, and there are three known isoforms of the AKT that are critical in the cardiovascular system. Upstream regulators of AKT in the cardiovascular system are platelet derived growth factor (PDGF), vascular endothelial growth factor (VEGF), epithelial growth factor (EGF), and basic fibroblast growth factor (bFGF). During angiogenesis and during endothelial cell migration (which is an important function of angiogenesis), AKT regulates the secretion of VEGF. AKT regulates the function through the Akt-PI3K pathway. Moreover, AKT can directly phosphorylate eNOS at S1197, which plays a major role in angiogenesis and vascular permeability.

Example I above documents the effect of ACNs and PAs on endothelial cell migration. ACNs are able to significantly inhibit the endothelial cell migration in a collective cell migration setup, while PAs have an opposite effect (especially at low concentrations), as they are able to significantly increase the speed of endothelial cells. Finally, combinations of both ACNs:PAs result in increased endothelial movement.

In this example, the effects of anthocyanin and phenolic acids, and their combinations, from wild blueberry powder (Vaccinum angustifolium) on angiogenesis were evaluated to determine whether they operate to alter angiogenesis. More specifically, ACNs, PAs and their combinations were evaluated to determine whether they affect in vitro angiogenesis, gene expression of AKT, eNOS and VEGF, and protein synthesis of VEGF after acute exposure to the above compounds.

Materials and Methods

Cell Culture

Human umbilical vein endothelial cells (HUV-EC-C [HUVEC] (ATCC® CRL-1730™)) were purchased from the American Type Culture Collection (ATCC®) Manassas, Va., USA. Human umbilical vein endothelial cells were maintained in F-12K medium (Kaighn's modification of Ham's F-12 medium) (ATCC® 30-2004™) with 10% fetal bovine serum (FBS) (ATCC® 30-2020™) and 1% penicillin-streptomycin solution (ATCC® 30-2300™), heparin 0.1 mg/mL (Sigma, H3149), and endothelial cell growth supplement (ECGS) 0.03 mg/mL (Sigma E2759). The culture vessel for the growth of the cells was the Corning® T-75 flask (catalog #3276). The culture conditions for the cell line was air 95%, carbon dioxide (CO2) 5%, 90% of relative humidity (RH), and temperature of 37° C.

Extraction of ACNs and PA Fractions from Wild Blueberry Powder

Wild blueberries (WB) were provided as a composite by Wyman's (Cherryfield, Me., USA) and processed following standard procedures to obtain a freeze-dried powder (FutureCeuticals, Momence, Ill., USA). Vacuum-packed plastic bags with the wild blueberry powder were stored at −20° C. until use. The wild blueberry powder had a total content of 1.5% w/w of anthocyanins, with malvidin-3-galactoside and peonidin-3-glucoside being the most abundant forms. From the freeze-dried wild blueberry powder, three fractions were isolated: 1. Phenolic-rich fraction (ethyl acetate soluble, containing mainly chlorogenic acid); 2. Anthocyanin-rich fraction (methanol soluble fraction, containing mainly anthocyanins); and 3. Water soluble fraction.

Extraction of ACNs and PAs from WB Powder

The extraction of ACNs and PAs from the WB powder was performed according to a previously reported method. The protocol followed for the extraction of the bioactive compounds is described in detail by Del Bo′ C, Cao Y, Roursgaard M, Riso P, Porrini M, Loft S, et al., Anthocyanins and phenolic acids from a wild blueberry (Vaccinium angustifolium) powder counteract lipid accumulation in THP-1-derived macrophages, Eur J Nutr 2015, doi:10.1007/s00394-015-0835-z, which is incorporated herein by reference. Determination of total phenolic concentration was conducted by the Folin-Ciocalteu method. For the determination of the anthocyanin fraction concentration, a pH differential method was used.

Analysis of ACNs and PA Fractions

Wild blueberry ACN and PA fraction concentration was determined by HPLC. The system consisted of an Alliance mod. 2695 (Water, Milford, Mass.) equipped with a mod. 2998 photodiode array detector (Waters).

Cell Proliferation and Cytotoxicity Assay

The cell growth curve experiment provided information on the doubling time of the HUVECs. The ACN and PA cytotoxicity assay was conducted so that the optimum concentration and exposure time of the active compounds was used for the rest of the experiments. Different concentrations (0.001 μg/mL-1000 μg/mL for ACNs and 0.001 μg/mL-500 μg/mL for the PAs) were tested. Moreover, different exposure times were tested (30 min, 1 h, 3 h, 6 h, 12 h, 24 h, 48 h, and 72 h). Cell proliferation and cytotoxicity were conducted by using the alamarBlue assay (Life Technologies, DAL1025). Before conducting the cell proliferation and cytotoxicity experiments, a standard curve of alamarBlue was generated. Cells for both cell proliferation and cytotoxicity assay were measured by using Synergy 2 multiwell plate reader (Bio-Tek Instruments Inc., Winooski, Vt.) with excitation/emission (530 nm-560 nm/590 nm).

Angiogenesis Assay (Tube Formation)

Critical concentrations of ACNs, PAs, and ACNs:PAs were 60 μg/mL for ACNs, 0.002 μg/mL, 60 μg/mL, and 120 μg/mL for PAs, and 8 μg/mL and 60 μg/mL for ACNs:PAs. Endothelial cells (1×10⁴ cells/well) were plated and cultured on Matrigel (BD Biosciences) that were applied on an IBIDI μ-slide Angiogenesis plate (Ibidi, Martinsried, Germany) and incubated at 37° C. and 30 min for gel construction. With this method, cells were induced to form capillary-like tubes. After exposing the cells to anthocyanin, phenolics, and combinations of both fractions, including a control (untreated cells), the effect on the tube formation was photographed by using an inverted phase-contrast optical microscope (Nikon, TS100) after 4 h based on the treatment. The number of meshes, total meshes area, number of nodes, number of master junctions, and total master segment length were measured and analyzed with the computer program Image J with the Angiogenesis Analyzer plugin.

Gene Expression, Real-Time RT-PCR Analysis

Endothelial cells were cultured and maintained as described previously. HUVECs were treated with ACNs, PAs, and combinations of both bioactive compounds for 2 h. mRNA was isolated using the RNeasy Kit (Qiagen) and DNase Digestion (Qiagen) was also used for RNA purification. QuantiTect Reverse Transcription Kit (Qiagen) was used for cDNA synthesis and removal of genomic DNA. A two-step RT-PCR was followed using a CFX96 (BioRad) PCR system. A 20 μl PCR reaction volume was performed using TaqMan gene expression master mix (Invitrogen) and TaqMan primers (Invitrogen): VEGFA (Hs00900055_m1), NOS3 (Hs01574665_m1), AKT1 (Hs00178289_m1), and GAPDH (Hs99999905_m1).

Western Blot Analysis

For the detection of AKT1, VEGF, eNOS, and β-tubulin, total protein was extracted from the cells in RIPA lysis and extraction buffer (Thermo Fisher, 89901) supplemented with protease/phosphatase inhibitor cocktail (Cell Signaling, 5872). Concentration of total protein was measured using BCA assay (Thermo Fisher, 23225). Total protein samples were resolved by 4-20% mini-protean TGX stain-free protein gel (BioRab, 4568094). After transfer using Trans-Blot Turbo System (BioRad, 1704150), LF-PVDF membranes (BioRad, 1704274) were blotted with anti-Akt1 (1:1000, Cell Signaling, 2938), anti-Phospho-Akt1 (1:1000, Cell Signaling, 9018), anti-Phospho-Akt1 (1:5000, Abcam, ab81283), anti-VEGF (1 μg/mL, BD Biosciences, 612392), anti-eNOS (1:1000, Cell Signaling, 9572), and anti-β-Tubulin (1:1000, Cell Signaling, 2128). Proteins were detected with antibodies specific for either mouse or rabbit IRDye® 800CW Goat anti-Mouse IgG, (1:15000, Li-COR, 925-32310), and IRDye® 800CW Goat anti-Rabbit IgG, (1:15000, LiCOR, 925-32211) using the Li-COR Odyssey imaging system (Li-COR Biosciences). Moreover, for western blot analysis, VEGFA positive control was used (Abcam, ab55566 and R&D Systems, 293-VE-010). All assays were repeated at least four times in independent experiments.

Statistical Analysis

Statistical analysis was performed using GraphPad Prism 6.0 software (GraphPad Software, San Diego, Calif.). For the tube formation/angiogenesis experiments, the data were analyzed by one-way ANOVA. For post-hoc comparisons, Fisher's least significant difference (LSD) test was used. Each experiment was repeated four times (n=4) and each independent sample per group was tested four times (n=4). For the gene expression and western blot experiments a two-tailed Mann-Whitney U-test was performed to compare the control to ACNs, while one-way ANOVA was used for the PAs and combination (ACNs:PAs) groups. For post-hoc comparisons, Fisher's test was used. For gene expression experiments the control included 10 replicates (n=10) and each treatment group (ACNs, PAs, and ACNs:PAs) included 10 replicates (n=10). Lastly, for western blot experiments, the control included 4 replicates (n=4) and each treatment group (ACNs, PAs, and ACNs:PAs) included 4 replicates (n=4). All data in the graphs are expressed as mean±SEM. A p-value of <0.05 was considered significant.

Results

Angiogenesis Assay

Tube formation in vitro assay was first standardized for the HUVEC cell line. After determining the appropriate cell density and incubation time, experiments were performed in quadruplicates for each treatment.

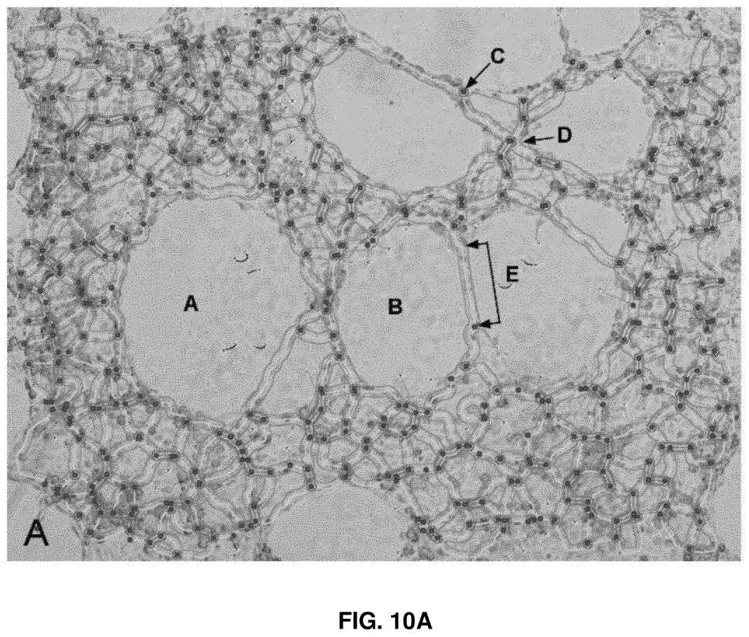

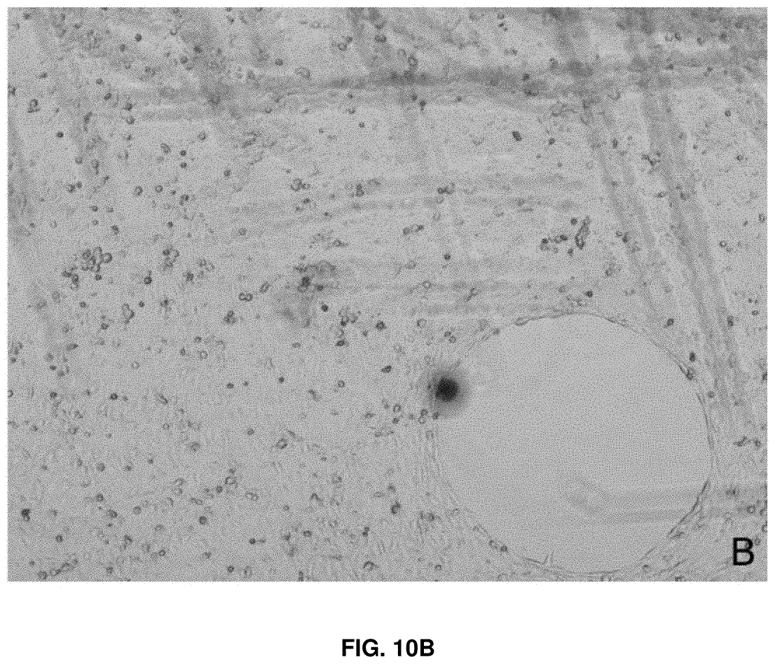

Pictures were captured after four hours of cells plating in the wells. All pictures were analyzed with Image J with the Angiogenesis Analyzer plugin (FIGS. 10A-10B). Cells treated with ACNs at 60 μg/mL were not able to form a complete tube network after four hours of incubation time (FIGS. 10A-10B).

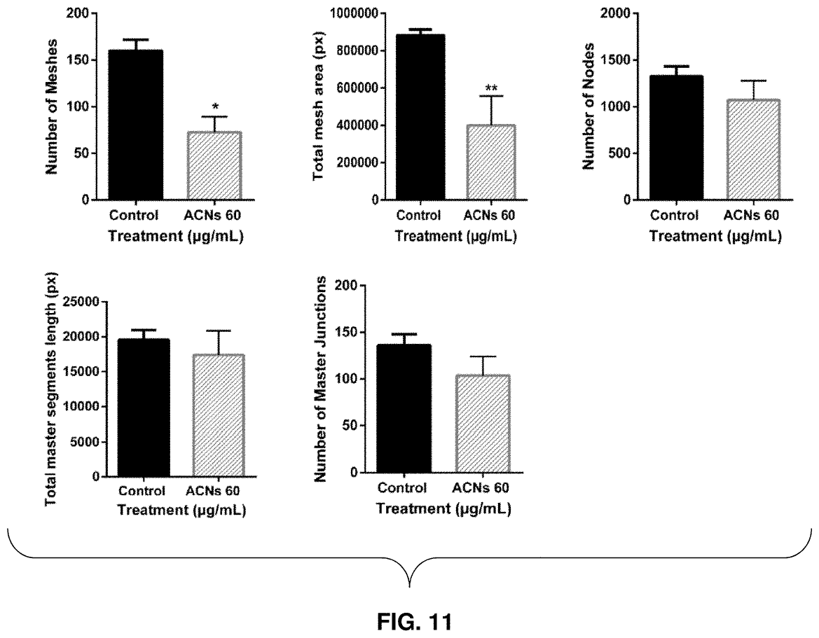

Only two parameters from the five tested had decreased numbers compared to the control (FIG. 11). However, the number of the meshes and the area of the mesh are of high importance for the formation of a complete endothelial tube network.

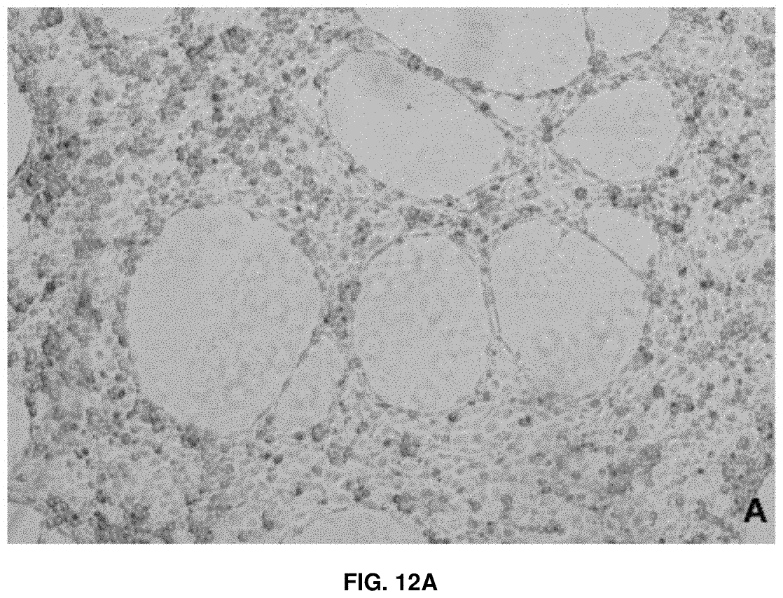

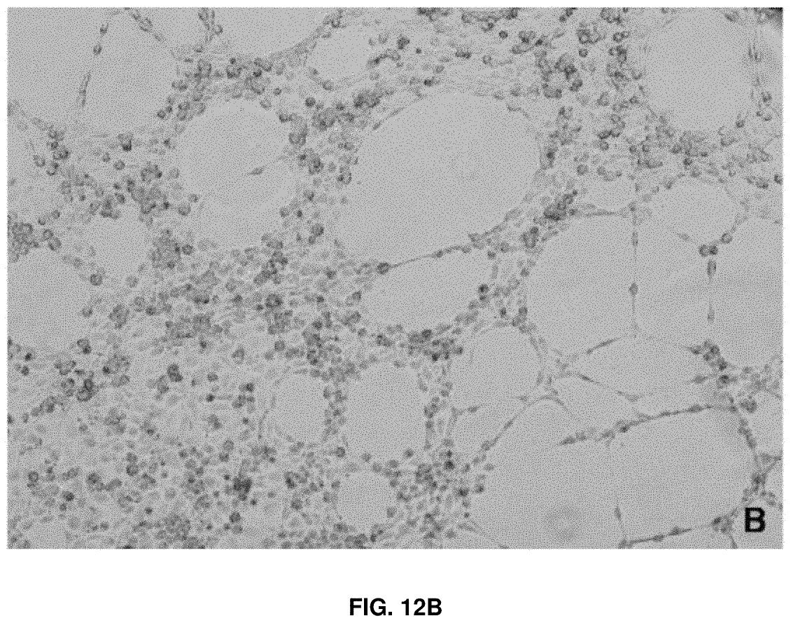

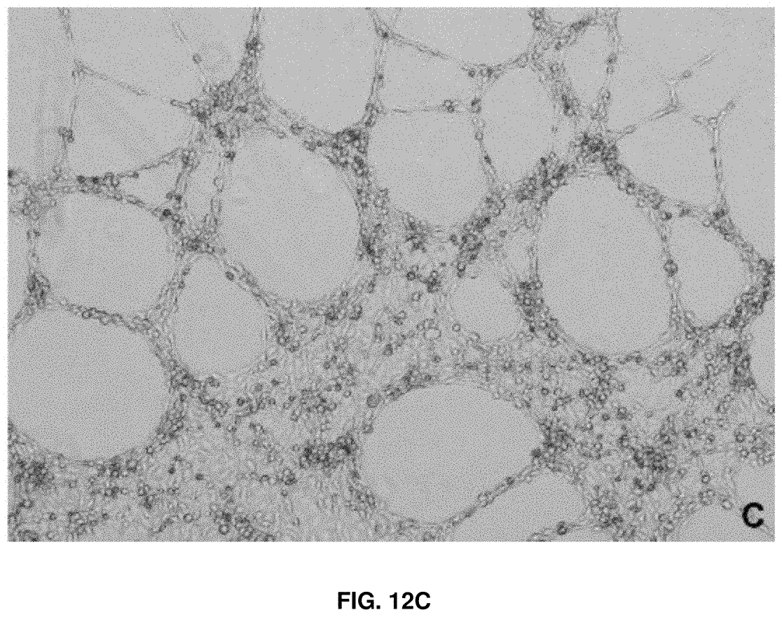

Cells treated with PAs at 0.002 μg/mL and 60 μg/mL were capable of forming a complete tube network (FIGS. 12A-12C). The tube network was also quantified for further analysis.

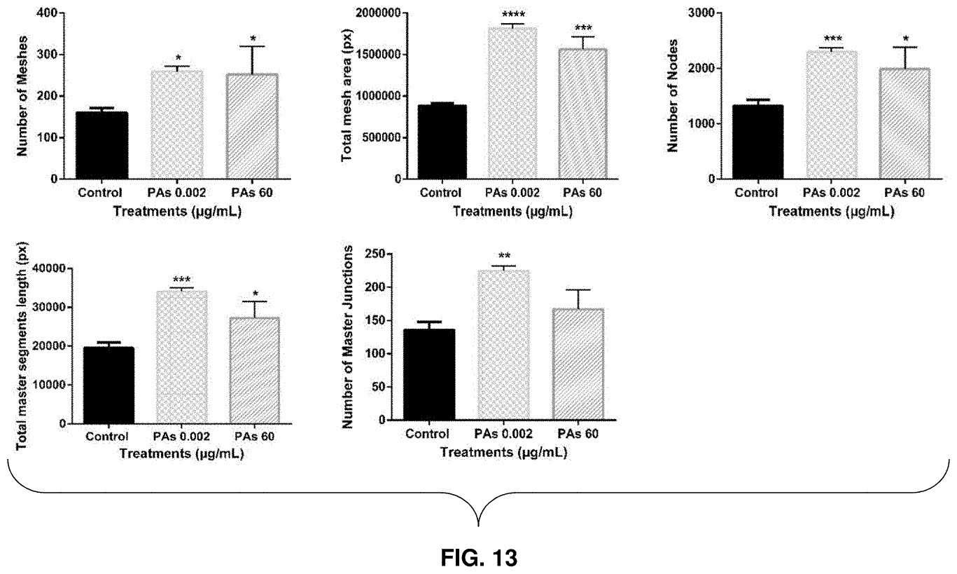

PAs at the lowest concentration tested documented the highest effect on the endothelial tube network, showing an increased number of meshes (*: p≤0.05), area of the mesh (****: p≤0.0001), number of nodes (***: p≤0.001), total master segment length (***: p≤0.001), and number of master junctions (**: p≤0.01) compared to the control (FIG. 13). Moreover, PAs at 60 μg/mL increased number of meshes (*: p≤0.05), area of the mesh (***: p≤0.001), number of nodes (*: p≤0.05), and number of master junctions (*: p≤0.05) compared to the control, while no effect was documented on the number of the master junctions (FIG. 13).

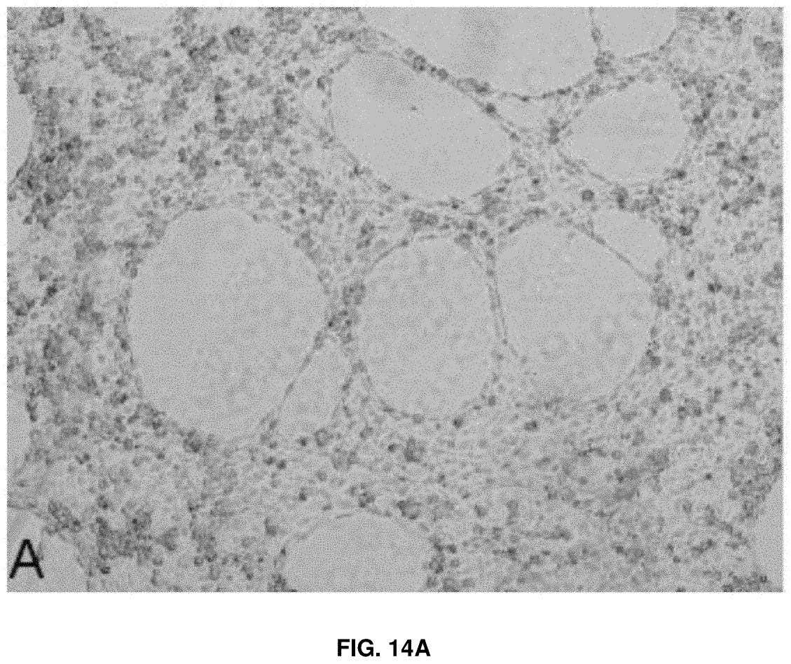

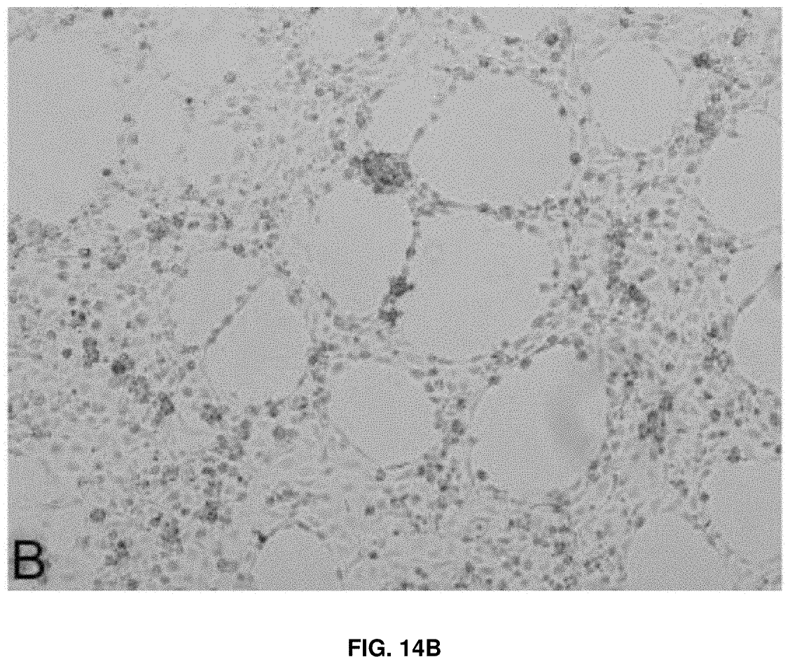

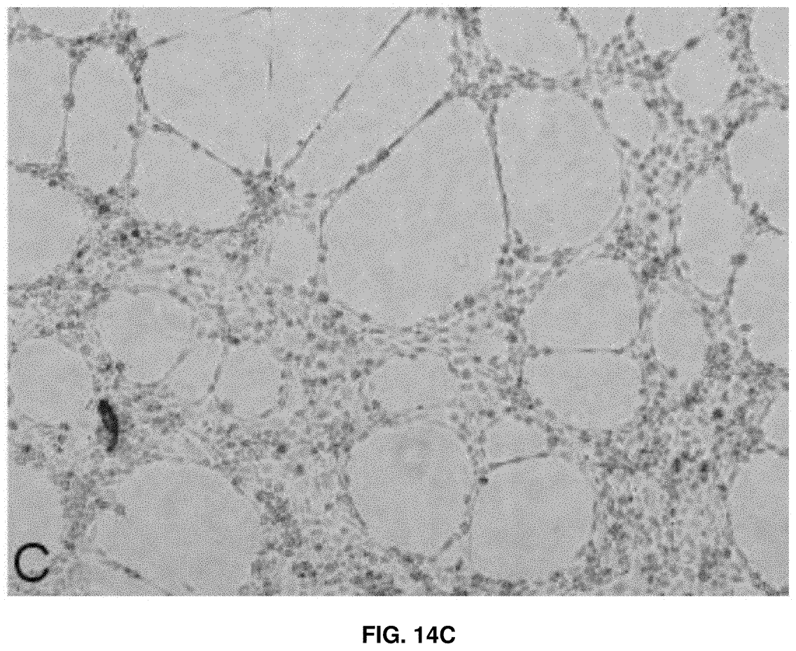

Finally, cells treated with a combination of both ACNs and PAs managed to form a complete endothelial network (FIGS. 14A-14C).

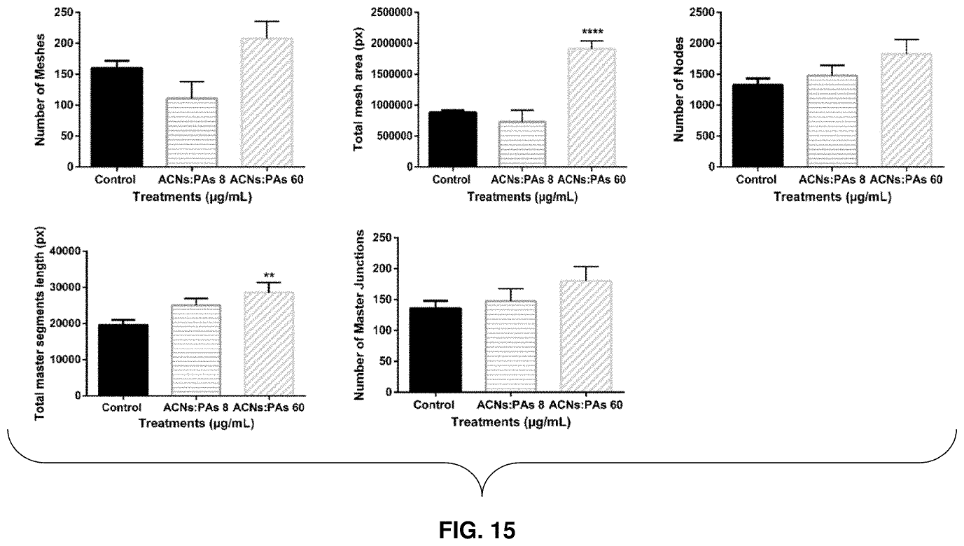

The combination of both ACNs and PAs at 8 μg/mL produced no statistical significant differences compared to the control on any of the five parameters of endothelial tube network integrity. However, ACNs:PAs at 60 μg/mL increased the total mesh area (****: p≤0.0001) compared to the control without having an effect on the number of meshes. Moreover, the total master segment length (**: p≤0.01) was increased compared to the control (FIG. 15).

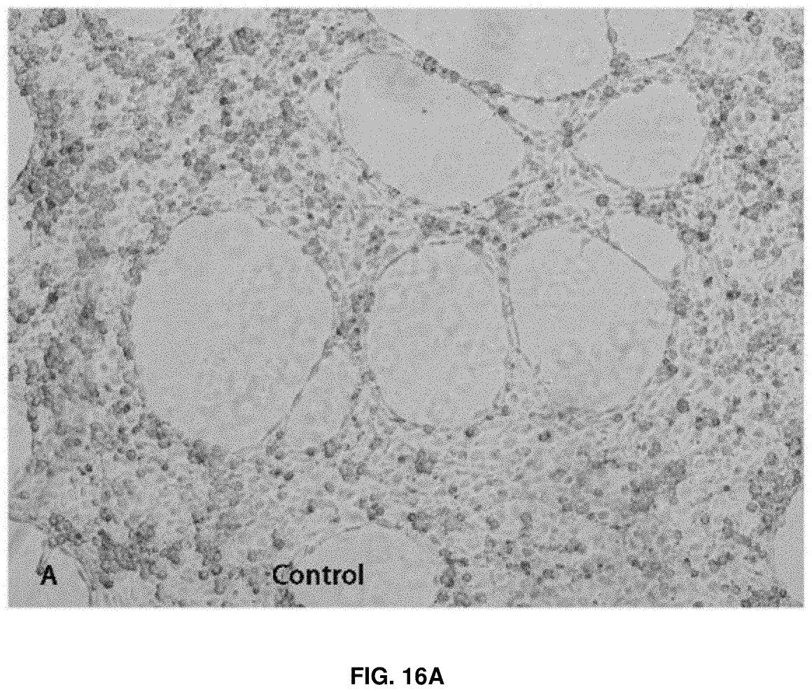

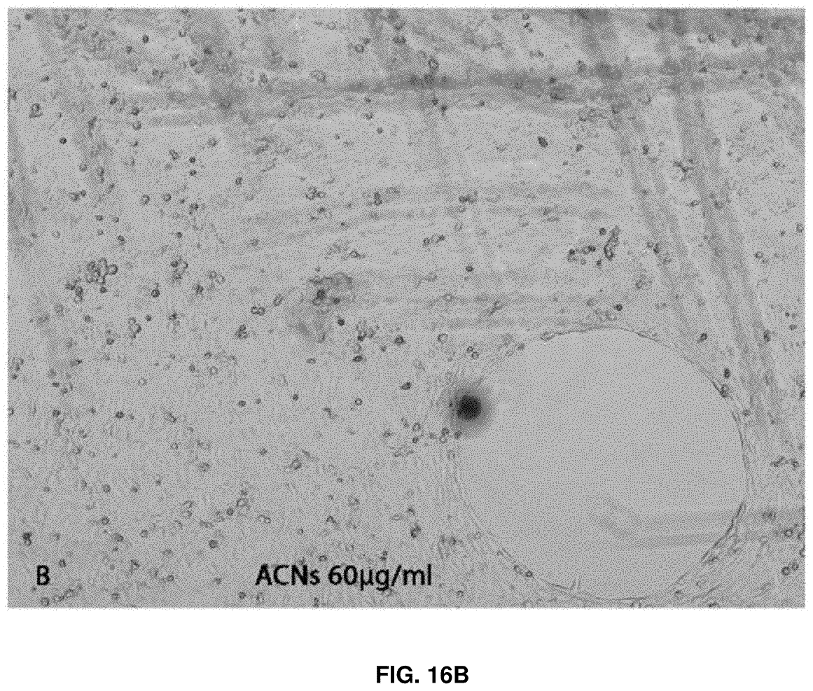

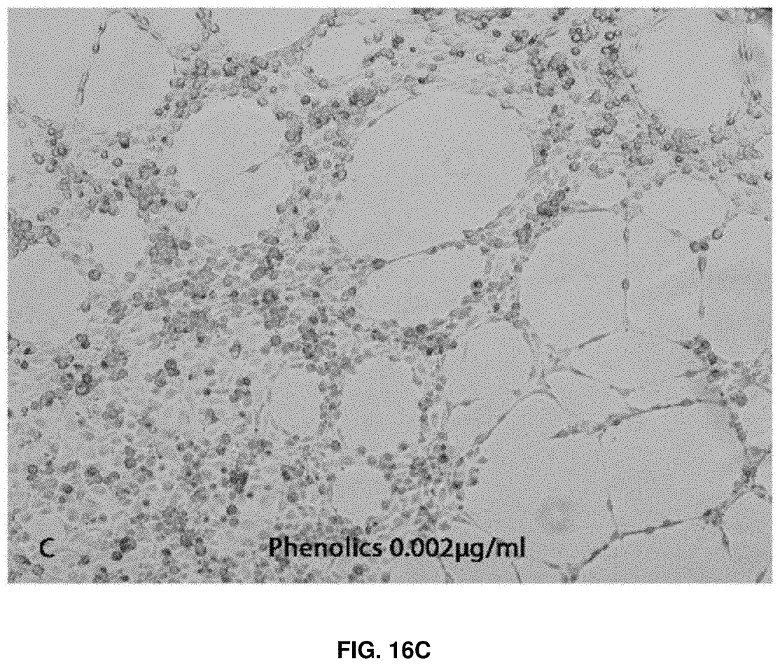

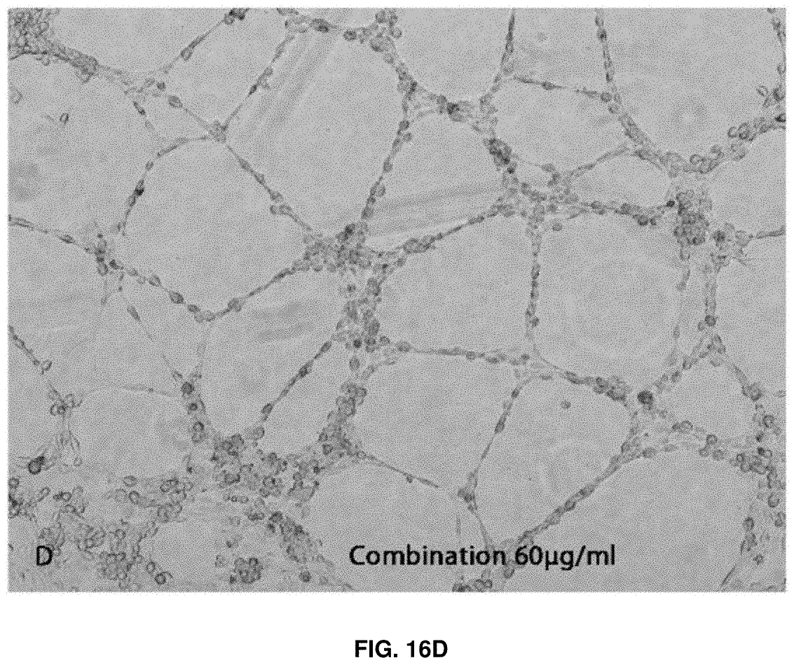

FIGS. 16A-16D show a summary from the tube formation assay from all treatments of ACNs, (60 μg/mL), PAs (60 μg/mL), and combinations of both fractions (60 μg/mL:60 μg/mL). The pictures in FIGS. 16A-16D were obtained after four hours of treatment.

Gene Expression

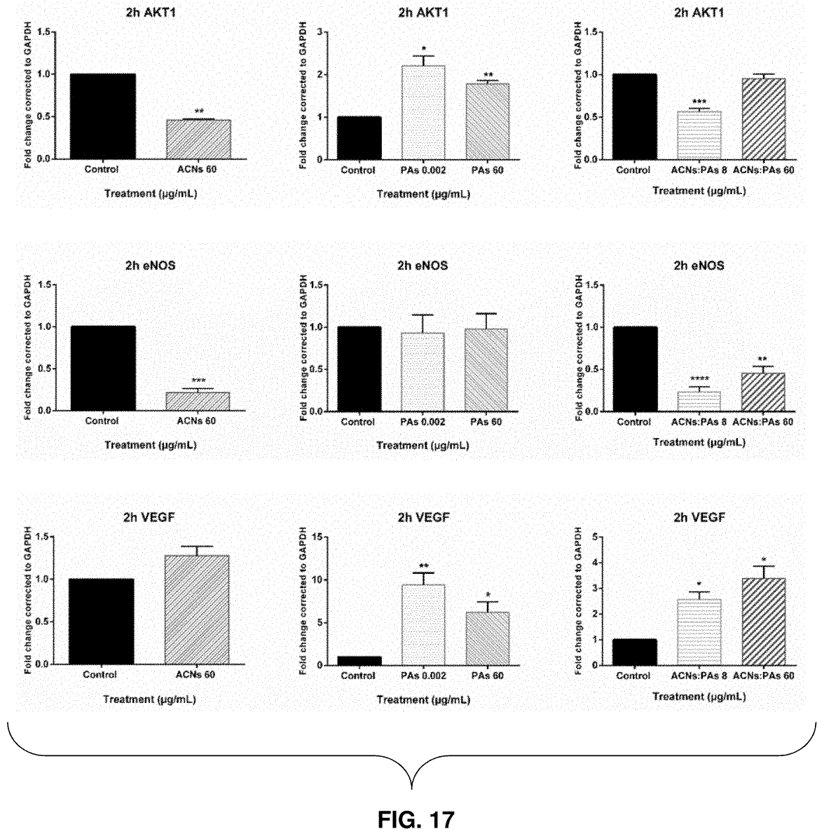

Gene expression was conducted for AKT1, eNOS, and VEGF (FIG. 17). Testing the gene expression of AKT1 for 2 hours post treatment resulted in all tested concentrations having a significant effect. When cells were treated with ACNs at 60 μg/mL, a reduced expression compared to the control resulted. PAs at 0.002 μg/mL and 60 μg/mL increased the expression at 2 hours compared to the control. Combinations of both fractions ACNs:PAs at 8 μg/mL resulted in a decreased gene expression for both time points while ACNs:PAs at 60 μg/mL had no effect at 2 hours.

Similarly, ACNs at 60 μg/mL documented reduced gene expression for eNOS at 2 hours. No effect was documented with PAs for both time points while ACNs:PAs at 8 μg/mL and 60 μg/mL resulted in decreased gene expression levels at 2 hours post treatment.

During treatment with ACNs 60 μg/mL, no effect on VEGF gene expression levels compared to the control was observed. Treatment with PAs at 0.002 μg/mL and 60 μg/mL for 2 hours resulted in an increased gene expression level compared to the control. Similar to the PAs, combinations at ACNs:PAs 8 μg/mL and ACNs:PAs 60 μg/mL resulted in increased gene expression levels compared to the control after 2 hours.

Western Blot Analysis

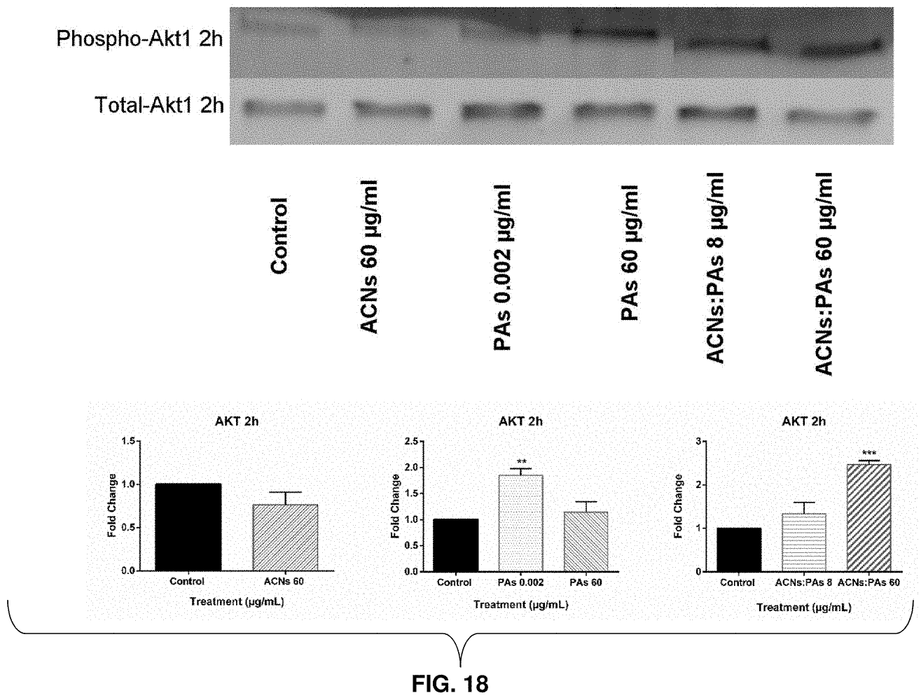

Western blot analysis for AKT1 (FIG. 18) documented that there are statistically significant differences compared to the control after 2 hours of treatment with PAs at 0.002 μg/mL (**: p≤0.01) and ACNs:PAs at 60 μg/mL (***: p≤0.001).

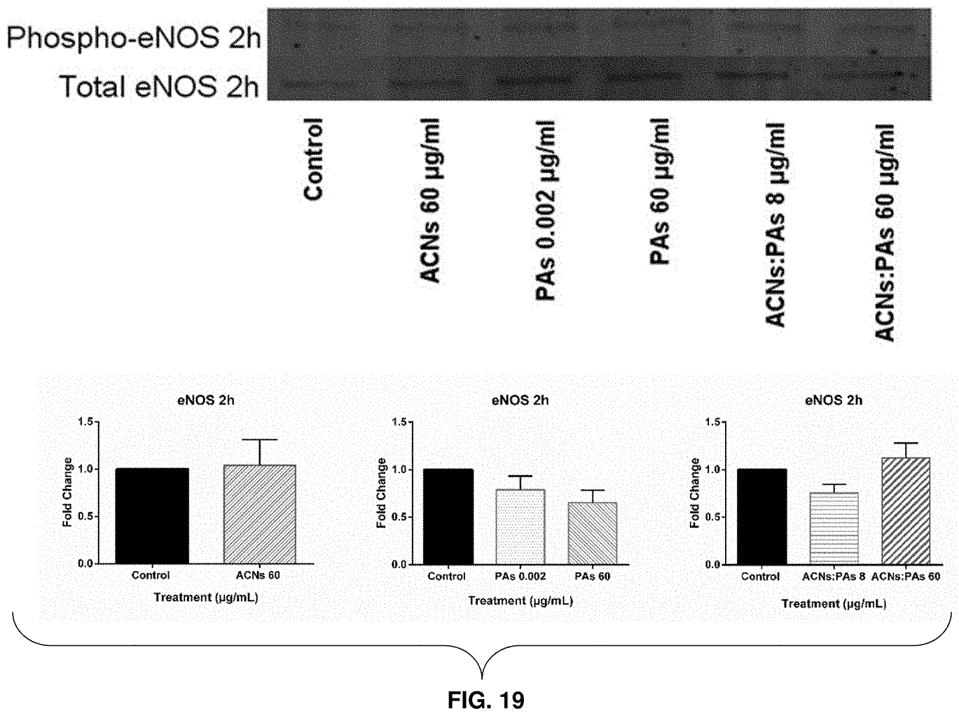

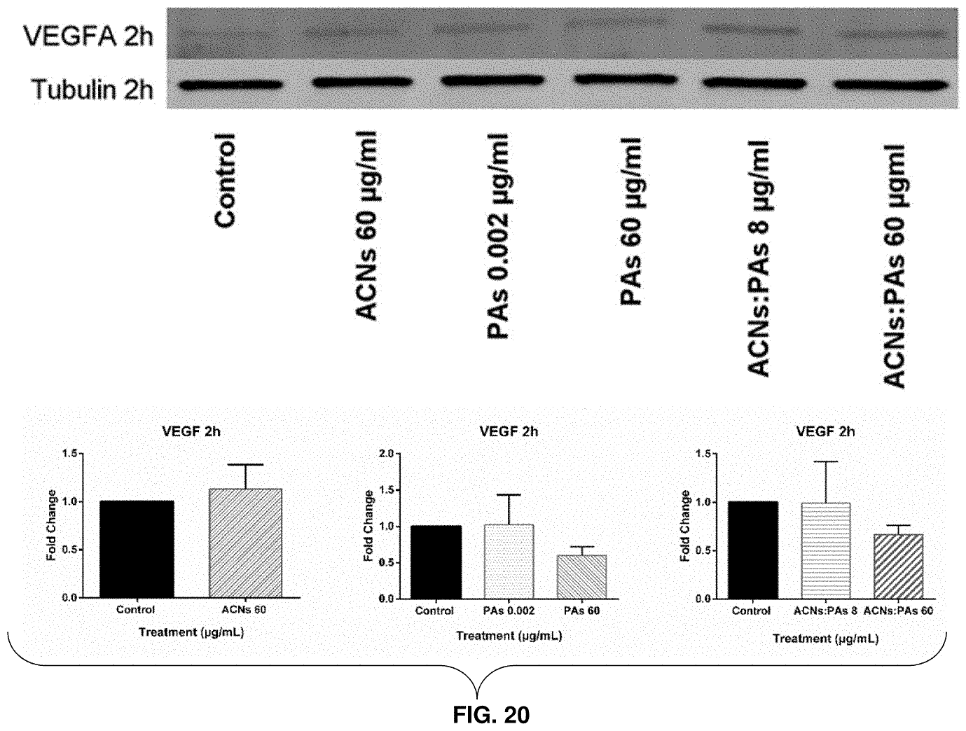

Western blot analysis for eNOS (FIG. 19) documented no statistically significant differences compared to the control after 2 hours of treatment with PAs. Similarly, the western blot analysis for VEGF (FIG. 20) documented no differences between control and PA treatments.

Discussion

This example demonstrates the effects of different fractions from the wild blueberries on endothelial in vitro tube formation assay and protein synthesis of AKT, eNOS, and VEGF. This example documented that different fractions at different concentrations have a different impact on the integrity of the endothelial network. The particular assay is simple, rapid, and reliable, can generate quantitative results, and is overall better than any other in vitro assay to assess angiogenic regulators.

This example shows that ACNs are able to inhibit cell migration while PAs and combinations of both fractions have the opposite results, inducing the speed of endothelial cell migration. Moreover, even low concentrations such as 0.002 μg/mL are sufficient to change the endothelial migration speed.

This example documents the effect of ACNs on angiogenesis through the tube formation assay. When HUVECs were treated with ACNs at 60 μg/mL, the number of meshes (*: p≤0.05) and the total area of the meshes (**: p≤0.01) were significantly reduced compared to the control with number of nodes, total master segment length, and number of master junctions remaining unaffected. The effects of ACNs on angiogenesis were documented through measurement of AKT, eNOS, and VEGF gene expression and protein synthesis. Anthocyanins were found to inhibit AKT (**: p≤0.01) and eNOS (***: p≤0.001) gene expression 2 h post treatment. Interestingly, ACNs had no significant effect on the VEGF gene expression. However, western blot analysis documented no significant results for all three markers. Changes in gene expression do not necessarily translate to increased protein levels since proteins can undergo posttranslational modification. The results in this example documented inhibition of angiogenesis with ACNs.

For phenolic acids, a different pattern from ACNs was documented. From the tube formation experiments, it was found that all parameters of angiogenesis (number of meshes, total area of mesh, number of nodes, total master segment length, and number of master junctions) were significantly increased compared to the control when HUVECs were treated with PAs at 0.002 μg/mL. The same results were documented at 60 μg/mL only without the significant effect on the number of the master junctions.

Following evaluation of PAs on angiogenesis through gene expression of AKT, eNOS, and VEGF, it was noted that gene expression was increased with PAs at 0.002 μg/mL (*: p≤0.05) and 60 μg/mL (**: p≤0.01) 2 h post treatment. Protein levels of AKT match the gene expression profile. Gene expression for eNOS documented no effect at 2 h. VEGF gene expression significantly increased its expression at 2 h 0.002 μg/mL (**: p≤0.01) and 60 μg/mL (*: p≤0.05), which did not translate to the protein level.

Angiogenesis assay results for the combination of both fractions documented only increased total mesh area and total master segment length when cells were treated with 60 μg/mL:60 μg/mL. No significant differences were documented for 8 μg/mL:8 μg/mL. Finally, combinations of both fractions documented decreased gene expression for AKT and eNOS, while increased expression was documented for VEGF at 2 h post treatment. Even though gene expression of AKT decreased compared to the control, increased protein synthesis was documented at 2 h post treatment at 60 μg/mL:60 μg/mL. Protein synthesis of eNOS agrees with the gene expression results while no effect was documented for VEGF protein levels.

The results from this example are different from known studies involving PAs, since increased angiogenesis and modulation of markers of angiogenesis such as AKT, eNOS, and VEGF under specific time and concentration parameters were observed. Moreover, this example evaluated chlorogenic acid since chlorogenic acid was predominant in the sample.

The present example examined the effects of wild blueberry fractions on HUVECs after acute exposure. A range of low and high concentrations of anthocyanins and phenolic acids was examined, as well as combinations of both, on cell cytotoxicity, angiogenesis, gene expression, and protein synthesis of AKT1, eNOS, and VEGF. Some of the concentrations used in the experiments (anthocyanins, phenolic acids, and combination at 0.002 μg/mL and 8 μg/mL) were close to the physiologically reported concentrations that have been observed in the blood stream.

The description of the various aspects and embodiments of the present invention has been presented for purposes of illustration and description. It is not intended to be exhaustive of all embodiments or to limit the invention to the specific aspects disclosed. Obvious modifications or variations are possible in light of the above teachings and such modifications and variations may well fall within the scope of the invention as determined by the appended claims when interpreted in accordance with the breadth to which they are fairly, legally and equitably entitled.

1. A method of modulating endothelial cell migration, the method comprising contacting endothelial cells with an effective amount of an extract of berries from the Vaccinium family to modulate endothelial cell migration.

2. The method of claim 1, wherein the berries are blueberries.

3. The method of claim 1, wherein the extract is a phenolic-rich fraction, and endothelial cell migration is increased.

4. The method of claim 3, wherein the phenolic-rich fraction comprises chlorogenic acid.

5. (canceled)

6. The method of claim 3, wherein the effective amount of the extract is a concentration ranging from about 0.002 μg/mL to about 120 μg/mL.

7. The method of claim 3, wherein the effective amount of the extract is a concentration of about 60 μg/mL.

8. The method of claim 1, wherein the extract is an anthocyanin-rich fraction, and endothelial cell migration is inhibited.

9. (canceled)

10. The method of claim 8, wherein the effective amount of the extract is a concentration of about 60 μg/mL.

11. The method of claim 1, wherein the extract is a phenolic-rich fraction combined with an anthocyanin-rich fraction.

12. (canceled)

13. The method of claim 11, wherein the effective amount of the extract is a concentration ranging from about 8 μg/mL:8 μg/mL of anthocyanin-rich fraction:phenolic-rich fraction to about 60 μg/mL:60 μg/mL of anthocyanin-rich fraction:phenolic-rich fraction.

14. The method according to claim 1, wherein the extract is an ethyl acetate soluble fraction.

15. The method according to claim 1, wherein the extract is a methanol soluble fraction.

16. The method of claim 1, wherein the extract is from V. angustifolium.

17. The method of claim 1, wherein the endothelial cells are human endothelial cells.

18. The method of claim 1, wherein angiogenesis or wound healing is modulated.

19. A method of modulating angiogenesis, the method comprising contacting endothelial cells with an effective amount of an extract of berries from the Vaccinium family and modulating angiogenesis.

20. A method of modulating gene expression of one or more of RAC1, RHOA, eNOS, AKT1, or VEGF in cells, the method comprising contacting cells with an effective amount of an extract of berries from the Vaccinium family and modulating gene expression of one or more of RAC1, RHOA, eNOS, AKT1, or VEGF in the cells.

21. The method of claim 20, wherein the cells are endothelial cells.

22. The method of claim 20, wherein the cells are human endothelial cells.

23. The method of claim 20, wherein the extract comprises a phenolic-rich fraction, and RAC1 gene expression is increased, RHOA gene expression is increased, AKT1 gene expression is increased, or VEGF gene expression is increased.

24. The method of claim 23, wherein the effective amount is about 0.002 μg/mL, about 60 μg/mL, or about 120 μg/mL.

25. The method of claim 20, wherein the extract comprises an anthocyanin-rich fraction, and RAC1 gene expression is increased, Of RHOA gene expression is increased, eNOS gene expression is decreased, or AKT1 gene expression is decreased.

26. The method of claim 25, wherein the effective amount is about 60 μg/mL.

27. The method of claim 20, wherein the extract comprises a combination of an anthocyanin-rich fraction and a phenolic-rich fraction, and RAC1 expression is increased, RHOA gene expression is increased, eNOS gene expression is decreased, or AKT1 gene expression is decreased.

28. The method of claim 27, wherein the effective amount is about 8 μg/mL or about 60 μg/mL, at a 1:1 ratio of anthocyanin-rich fraction to phenolic-rich fraction.

29-41. (canceled)

42. The method of claim 23, wherein protein expression of AKT1 is increased.

43-55. (canceled)
